SEQUENCE LISTING (5 sequences)

SEQ ID NO: 1 - DNA, 6669 bp, Artificial Sequence
  shTACE
gaattcgcggccctagcttgggatctttgtgaaggaaccttacttctgtggtgtgacataattggacaaactacctacagagatttaaagctctaaggtaaatataaaatttttaagtgtataatgtgttaaactagctgcatatgcttgctgcttgagagttttgcttactgagtatgatttatgaaaatattatacacaggagctagtgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgatcctgcattaatgaatcggccaacgcgcggggagaggcggtttgcgtattggctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggaaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgaacctggtgcatgacccgcaagcccggtgcctgagtttaacgaaatgaccgaccaagcgacgcccaacctgccatcacgatggccgcaataaaatatctttattttcattacatctgtgtgttggttttttgtgtggatcgatagcgataaggatcgatccgcgcatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaccgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagagcttgcaattcgcgcgtttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtagtacgaggccctttcactcattagatgcatgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagcctaggccgcggaccatgtccggcttgaacgacatcttcgaggcccagaagatcgagtggcacgaggaaaagcttcgaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagctcgagtgcggccccaaataatgattttattttgactgatagtgacctgttcgttgcaacaaattgatgagcaatgcttttttataatgccaactttgtacaaaaaagcaggctgcgatcgctcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaggatccgacacctgctgcaatagtgatcaagagtcactattgcagcaggtgtttttttg

SEQ ID NO: 2 - DNA, 20 bp, Artificial Sequence
  TACE Forward primer
gtacgtcgatgcagagcaaa

SEQ ID NO: 3 - DNA, 20 bp, Artificial Sequence
  TACE Reverse primer
aaaccagaacagacccaacg

SEQ ID NO: 4 - protein, 11 aa, Artificial Sequence
  peptide repeating unit of PAs-s
CRRRRRRRRRC

SEQ ID NO: 5 - protein, 26 aa, Artificial Sequence
  gene carrier
CDDDDDDDDRRRRRRRRRRRRRRRRC

(19) United States
(12) Patent Application Publication
(10) Pub. No.: US 2018/0193474 A1
(43) Pub. Date: Jul. 12, 2018

(54) Gene/carrier complex for preventing or treating inflammatory diseases

(71) Applicants: INDUSTRY-UNIVERSITY COOPERATION FOUNDATION HANYANG UNIVERSITY, Seoul (KR); INDUSTRY-UNIVERSITY COOPERATION FOUNDATION HANYANG UNIVERSITY ERICA CAMPUS, Gyeonggi-do (KR)

(72) Inventors: Yong-Hee KIM, Seoul (KR); Chul-Su YANG, Gyeonggi-do (KR); Yoonsung SONG, Gyeonggi-do (KR); So Mi KIM, Seoul (KR); Ye-Ram KIM, Daejeon (KR); Jee-Young CHUNG, Seoul (KR); Qurrat UI AIN, Seoul (KR)

(21) Appl. No.: 15/721,199

(22) Filed: Sep. 29, 2017

(30) Foreign Application Priority Data
Sep. 30, 2016 (KR) 10-2016-0126284
Sep. 25, 2017 (KR) 10-2017-0123413
Sep. 25, 2017 (KR) 10-2017-0123414

(51) Int. Cl. A61K 47/64; A61K 31/7105; A61K 48/00; A61P 37/06; A61K 47/56; A61K 47/18; A61K 31/738

(52) U.S. Cl. CPC A61K 47/6455; A61K 31/7105; A61K 48/0041; A61K 31/738; A61P 37/06; A61K 47/56; A61K 47/183; A61K 48/0066

(57) ABSTRACT

Disclosed is a gene/carrier complex for preventing or treating inflammatory diseases, including tumor necrosis factor-α converting enzyme (TNF-α converting enzyme, TACE) shRNA and a nonviral gene carrier, wherein the nonviral gene carrier includes an acetate of disulfide-linked poly(oligo-arginine) or a TFA salt of poly(oligo-aspartic acid)poly(oligo-arginine).

### BACKGROUND

### 1. Field of the Invention

The prevent invention relates to a gene/carrier complex for preventing or treating inflammatory diseases.

### 2. Discussion of Related Art

The expression of tumor necrosis factor-α converting enzyme (TACE) is increased in various inflammatory diseases, such as rheumatoid arthritis, acute lung injury, and inflammatory bowel disease. As a result, an excessive increase in the expression of TACE leads to an increase in the level of tumor necrosis factor-α (TNF-α) and activation of the inflammatory signal system, resulting in worsening of inflammatory diseases. Therefore, it is possible to prevent and treat various inflammatory diseases by inhibiting TACE expression by introducing gene therapy agents. Use of small interfering RNA (siRNA) or short hairpin RNA (shRNA) targeting TACE is a method to lower TACE expression at the cellular level.

To obtain shRNA targeting TACE (hereinafter, referred to as “shTACE”), a therapeutic gene construct, E. coli is transformed with the desired gene and cultured at 37° C. After culture, the transformed E. coli is lysed to isolate the desired gene. The process of obtaining the desired gene by lysing E. coli (i.e., Prep) is classified as Miniprep, Maxiprep, and the like according to yield and experimental procedure.

In the case of shTACE, a therapeutic gene construct, described in Non-Patent Document 1 (SOMI Kim, Master's Thesis, Graduate School of Hanyang University (February 2014), “Development of non-viral RNA interference system against TACE (Tumor necrosis factor-α converting enzyme) for the treatment of inflammatory diseases Development of nonviral gene delivery system that interferes with expression of tumor necrosis factor-α converting enzyme (TACE) for treating inflammatory diseases”) previously reported by the present inventors, even with Maxiprep, the degree of amplification of shTACE is very low, resulting in a very low yield of shTACE. Accordingly, to amplify shTACE having an existing sequence described in Non-Patent Document 1, which is difficult to amplify using Maxiprep, it is inevitable to use Miniprep. However, the disadvantages of using Miniprep are as follows. First, gene yield obtained by Miniprep is much lower than gene yield obtained by general Maxiprep and the like. Since the yield is low when using Miniprep, Miniprep should be repeated to obtain the amount of DNA needed for experimentation. This makes the experiments cumbersome and costly. In addition, since endotoxins are not removed through the Miniprep procedure, additional experiments should be performed to remove endotoxins after obtaining genes, which inconveniences experimenters. These additional processes lower the yield and purity of the obtained gene. In particular, in the case that endotoxins are lipopolysaccharides (LPSs) found in the outer membrane of gram-negative bacteria, strong inflammatory responses may be caused in animals. Thus, endotoxins must be removed before performing animal experiments.

On the other hand, the Maxiprep procedure automatically removes endotoxins, and thus no additional procedures are required to remove endotoxins. Thus, when using Maxiprep, the yield and purity of the obtained gene may be improved. Development of a method of obtaining therapeutic gene constructs using the Maxiprep procedure is required.

In addition, since shTACE has low stability in the human body, there are limitations in applying shTACE to the human body. One goal of gene therapy is to deliver gene therapy agents into the nucleus efficiently through the cell membrane and nuclear membrane. Therefore, it is essential to study efficient gene delivery systems for gene therapy.

Accordingly, the present inventors prepared a novel gene therapy agent represented by SEQ ID NO: 1, in which the sequence of a conventional TNF-α converting enzyme (TACE) shRNA was modified, and synthesized a novel carrier capable of stably delivering the gene therapy agent. Thus, the present invention was completed by developing the gene therapy agent (gene/carrier complex) having an excellent effect on preventing or treating inflammatory diseases.

### SUMMARY OF THE INVENTION

Therefore, it is an objective of the present invention to provide a gene/carrier complex including tumor necrosis factor-α converting enzyme (TNF-α converting enzyme, TACE) shRNA represented by SEQ ID NO: 1 and a nonviral gene carrier.

It is another objective of the present invention to provide a composition for preventing or treating inflammatory diseases, including the gene/carrier complex as an active ingredient.

### BRIEF DESCRIPTION OF THE DRAWINGS

The above and other objects, features and advantages of the present invention will become more apparent to those of ordinary skill in the art by describing in detail exemplary embodiments thereof with reference to the attached drawings, in which:

FIG. 1 is an electrophoresis image showing whether a complex was formed between the novel TNF-α converting enzyme (TACE) shRNA (shTACE) and the nonviral gene carrier (hereinafter, referred to as “PAs-s”) including the acetate of disulfide-linked poly(oligo-arginine) according to the present invention, depending on the weight ratio of PAs-s to shTACE;

FIG. 2 is a graph showing the zeta potential of the novel shTACE/PAs-s complex according to the present invention, depending on the weight ratio of PAs-s to shTACE;

FIG. 3 is a graph showing the particle size of the novel shTACE/PAs-s complex according to the present invention, depending on the weight ratio of PAs-s to shTACE;

FIG. 4 is a graph showing the polydispersity index (PDI) values of the novel shTACE/PAs-s complex according to the present invention, depending on the weight ratio of PAs-s to shTACE;

FIG. 5 is a graph showing the cytotoxicity of the novel shTACE/PAs-s complex according to the present invention, depending on the weight ratio of PAs-s to shTACE;

FIG. 6 is a graph showing the cytotoxicity of the novel shTACE/PAs-s complex according to the present invention, depending on the concentration of the novel shTACE/PAs-s complex in which the weight ratio of PAs-s to shTACE is optimal;

FIG. 7 is a graph showing the cytotoxicity of each complex after treatment with an existing shTACE/rPOA complex or the novel shTACE/PAs-s complex;

FIG. 8 is a graph showing TNF-α converting enzyme (TACE) mRNA levels in cells treated with the existing shTACE/rPOA complex or the novel shTACE/PAs-s complex. After cells were treated with the existing shTACE/rPOA complex and the novel shTACE/PAs-s complex, respectively, TACE mRNA levels were compared;

FIG. 9 is a graph showing change in body weight after treatment with the existing shTACE/rPOA complex or the novel shTACE/PAs-s complex;

FIG. 10 is an image showing liver tissue and spleen tissue after treatment with the existing shTACE/rPOA complex or the novel shTACE/PAs-s complex;

FIG. 11 is a graph showing the results of measuring the size of the novel shTACE/PAs-s complex over time depending on the presence or absence of treatment with a reducing agent;

FIG. 12 is a graph showing the transfection efficiency of a PAs-s gene carrier. The transfection efficiency was evaluated by measuring the expression level of a luciferase plasmid;

FIG. 13 is a graph showing the effect of the novel shTACE/PAs-s complex on the level of TACE mRNA. After inducing in vitro inflammation with lipopolysaccharide (LPS) treatment, cells were treated with the shTACE/PAs-s complex and the level of TACE mRNA was measured;

FIG. 14 is a graph showing the effect of the novel shTACE/PAs-s complex on the level of TNF-α. After inducing in vitro inflammation with lipopolysaccharide (LPS) treatment, cells were treated with various concentrations of the novel shTACE/PAs-s complex and a monoclonal antibody, and the level of TNF-α, an inflammatory cytokine, was measured using ELISA;

FIG. 15 includes an experimental scheme and images showing the in vivo effect of the novel shTACE/PAs-s complex on the level of TACE protein. The novel shTACE/PAs-s complex was administered to animals. Six and eleven days after administration, the amount of TACE protein in liver and spleen was measured;

FIG. 16 includes an experimental scheme and images showing the effect of the novel shTACE/PAs-s complex on the level of TACE protein in an animal model of ulcerative colitis. After the novel shTACE/PAs-s complex was administered to animals, ulcerative colitis was induced in the animals. Then, in the animal model of ulcerative colitis, the amount of TACE protein in intestines was measured;

FIG. 17 shows the schedule of shTACE/PAs-s complex administration and animal modeling for ulcerative colitis to confirm the ulcerative colitis prevention effect of the shTACE/PAs-s complex in an animal model of ulcerative colitis;

FIG. 18 is a graph showing the effect of the shTACE/PAs-s complex on survival. The shTACE/PAs-s complex was administered to animals prior to animal modeling for ulcerative colitis, and survival rate was measured;

FIG. 19 is a graph showing the effect of the shTACE/PAs-s complex on body weight. The shTACE/PAs-s complex was administered to animals prior to animal modeling for ulcerative colitis, and body weight was measured;

FIG. 20 is a graph showing the effect of the shTACE/PAs-s complex on colitis scores. The shTACE/PAs-s complex was administered to animals prior to animal modeling for ulcerative colitis, and colitis scores were measured;

FIG. 21 includes a graph showing the effect of the shTACE/PAs-s complex on colon length and an image showing the representative colons of each test group. The shTACE/PAs-s complex was administered to animals prior to animal modeling for ulcerative colitis, and colon images were obtained from the animals and colon length was measured;

FIG. 22 shows a histological analysis. The shTACE/PAs-s complex was administered to animals prior to animal modeling for ulcerative colitis, and a histological analysis was performed;

FIG. 23 is a graph showing the effect of the shTACE/PAs-s complex on the activity of myeloperoxidase (MPO). The shTACE/PAs-s complex was administered to animals prior to animal modeling for ulcerative colitis, and MPO activity was measured;

FIG. 24 is a graph showing the effect of the shTACE/PAs-s complex on the activity of NADPH oxidase. The shTACE/PAs-s complex was administered to animals prior to animal modeling for ulcerative colitis, and the amount of intracellular reactive oxygen species indicating the activity of NADPH oxidase was measured;

FIG. 25 includes graphs showing the effect of the shTACE/PAs-s complex on the levels of inflammatory cytokines. The shTACE/PAs-s complex was administered to animals prior to animal modeling for ulcerative colitis, and IL-1β, TNF-α, and IL-6 levels were measured using ELISA;

FIG. 26 includes images showing the effect of the shTACE/PAs-s complex on the expression levels of proteins associated with the inflammatory signaling system. The shTACE/PAs-s complex was administered to animals prior to animal modeling for ulcerative colitis. The expression level of TACE was measured to determine whether the shTACE/PAs-s complex was functioning normally, and the expression levels of proteins associated with the inflammatory signaling system, including phosphorylated ERK1/2, phosphorylated p38, IκB, COX-2, and iNOS were measured;

FIG. 27 shows the schedule of shTACE/PAs-s complex administration and animal modeling for ulcerative colitis to confirm therapeutic effect of the shTACE/PAs-s complex in an animal model of ulcerative colitis;

FIG. 28 is a graph showing the effect of the shTACE/PAs-s complex on survival rate. The shTACE/PAs-s complex was administered to an animal model of ulcerative colitis, and survival rate was measured;

FIG. 29 is a graph showing the effect of the shTACE/PAs-s complex on body weight. The shTACE/PAs-s complex was administered to an animal model of ulcerative colitis, and body weight was measured;

FIG. 30 is a graph showing the effect of the shTACE/PAs-s complex on colitis scores. The shTACE/PAs-s complex was administered to an animal model of ulcerative colitis, and colitis scores were measured;

FIG. 31 shows a histological analysis. The shTACE/PAs-s complex was administered to an animal model of ulcerative colitis, and a histological analysis was performed;

FIG. 32 includes images showing the effect of the shTACE/PAs-s complex on the expression levels of proteins associated with the inflammatory signaling system. The shTACE/PAs-s complex was administered to an animal model of ulcerative colitis, and the expression level of TACE protein and the expression levels of proteins associated with the inflammatory signaling system, including IκB, COX-2, and iNO were measured;

FIG. 33 includes images showing the distribution of the shTACE/PAs-s complex in colon tissues. After intravenous administration of the shTACE/PAs-s complex, the distribution of the complex in the colon tissues was observed periodically;

FIG. 34 shows the schedule of rheumatoid arthritis modeling using DBA/1 mice and the schedule of shTACE/PAs-s complex administration;

FIG. 35 includes images showing the effect of the shTACE/PAs-s complex on the shape of the fingers and toes of experimental animals. After administering the shTACE/PAs-s complex to the animal model, images of the fingers and toes of the experimental animals were obtained;

FIG. 36 shows the results of H&E staining of tissue sections. After administering the shTACE/PAs-s complex to an animal model of rheumatoid arthritis, H&E staining was performed using tissues obtained from the animal model;

FIG. 37 shows the effect of the shTACE/PAs-s complex on inflammation and cartilage destruction. After administering the shTACE/PAs-s complex to an animal model of rheumatoid arthritis, the extent of inflammation and cartilage destruction in tissues were scored, and the results were tabulated;

FIG. 38 is an electrophoresis image showing whether a complex was formed between shTACE and a nonviral gene carrier (hereinafter, referred to as 8D9R) including the TFA salt of Cys-(8×Asp)-(9×Arg)-Cys, depending on the weight ratio of 8D9R to shTACE [NAKED shTACE: without 8D9R carrier, WW10: 8D9R/shTACE=10, WW15: 8D9R/shTACE=15, WW20: 8D9R/shTACE=20, WW25: 8D9R/shTACE=25, WW30: 8D9R/shTACE=30, and WW35: 8D9R/shTACE=35];

FIG. 39 is a graph showing the zeta potential of the shTACE/8D9R complex depending on the weight ratio of 8D9R to shTACE [WW20: 8D9R/shTACE=20, WW25: 8D9R/shTACE=25, and WW30: 8D9R/shTACE=30];

FIG. 40 includes graphs showing zeta potential distribution at a specific weight of the shTACE/8D9R complex [WW3: 8D9R/shTACE=3 and WW25: 8D9R/shTACE=25];

FIG. 41 is a graph showing the particle size of the shTACE/8D9R complex depending on the weight ratio of 8D9R to shTACE [WW20: 8D9R/shTACE=20, WW25: 8D9R/shTACE=25, and WW30: 8D9R/shTACE=30];

FIG. 42 is a graph showing the transfection efficiency of 8D9R as a peptide carrier in mouse-derived macrophages. The transfection efficiency was evaluated by measuring the expression level of a luciferase plasmid [PEI: polyethylenimine];

FIG. 43 is a graph showing the effect of the shTACE/8D9R complex on inflammation scores. The shTACE/8D9R complex was administered to experimental mice that had undergone a rheumatoid arthritis modeling process, and a histological analysis was performed to score inflammation;

FIG. 44 is a graph showing the effect of the shTACE/8D9R complex on cartilage destruction. The shTACE/8D9R complex was administered to experimental mice that had undergone a rheumatoid arthritis modeling process, and the extent of cartilage destruction was evaluated by histological analysis. The results are shown as scores;

FIG. 45 is an electrophoresis image showing whether a complex was formed between shTACE and a nonviral gene carrier (hereinafter, referred to as 8D16R) including the TFA salt of Cys-(8×Asp)-(16×Arg)-Cys, depending on the weight ratio of 8D16R to shTACE [ND: Naked shTACE (without 8D16R), 0.5: 8D16R/shTACE=0.5, 1: 8D16R/shTACE=1, 2: 8D16R/shTACE=2, 3: 8D16R/shTACE=3, and 4: 8D16R/shTACE=4];

FIG. 46 shows the zeta potential, particle size, and PDI value of the shTACE/8D16R complex when the weight ratio of 8D16R to shTACE is 4 (8D16R/shTACE=4);

FIG. 47 is a graph showing the cytotoxicity of the shTACE/8D16R complex in mouse-derived macrophages;

FIG. 48 is a graph showing the transfection efficiency of 8D16R as a peptide carrier in mouse-derived macrophages. The transfection efficiency was evaluated by measuring the expression level of a luciferase plasmid;

FIG. 49 is a graph showing the effect of the shTACE/8D16R complex on the level of TACE mRNA. Mouse-derived macrophages were treated with the shTACE/8D16R complex, and the level of TACE mRNA was measured;

FIG. 50 is a graph showing the effect of the shTACE/8D16R complex on the mRNA level of TNF-α, an inflammatory cytokine. Mouse-derived macrophages were treated with the shTACE/8D16R complex, and the level of TNF-α mRNA was measured;

FIG. 51 is a graph showing the effect of the shTACE/8D16R complex on the protein level of TNF-α, an inflammatory cytokine. Inflammation-induced mouse-derived macrophages were treated with the shTACE/8D16R complex, and the level of TNF-α protein was measured;

FIG. 52 shows the schedule of experimental animal modeling for rheumatoid arthritis and shTACE/8D16R complex administration;

FIG. 53 includes images showing the effect of the shTACE/8D16R complex on the shape of the fingers and toes of an animal model of rheumatoid arthritis. After administering the shTACE/8D16R complex to the animal model, images of the fingers and toes of the experimental animals were obtained;

FIG. 54 shows the results of H&E staining of tissue sections. After administering the shTACE/8D16R complex to an animal model of rheumatoid arthritis, H&E staining was performed using tissues obtained from the animal model;

FIG. 55 is a graph showing the effect of the shTACE/8D16R complex on the degree of inflammation progression (indicated by inflammation scores) in experimental mice that had undergone a rheumatoid arthritis modeling process. The shTACE/8D16R complex was administered to the experimental mice, and a histological analysis was performed to score inflammation;

FIG. 56 is a graph showing the effect of the shTACE/8D16R complex on cartilage destruction in experimental mice that had undergone a rheumatoid arthritis modeling process. The shTACE/8D16R complex was administered to the experimental mice, and the extent of cartilage destruction was analyzed; and

FIG. 57 is an image showing the targeting ability of an 8D16R carrier to bone resorption sites in experimental mice that had undergone a rheumatoid arthritis modeling process.

### DETAILED DESCRIPTION OF EXEMPLARY EMBODIMENTS

Hereinafter, the present invention will be described with reference to examples and comparative examples in detail. However, the present invention is not limited to these examples.

The present invention relates to a gene/carrier complex including tumor necrosis factor-α converting enzyme (TNF-α converting enzyme, TACE) shRNA and a nonviral gene carrier.

Hereinafter, the present invention is described in detail as follows.

The present inventors developed a novel shTACE, in which the sequence of a conventional shTACE was modified, to solve the problems of the conventional shTACE. The novel gene construct may be applied to both Miniprep and Maxiprep. The novel gene construct has excellent amplification rate when using Miniprep and Maxiprep, and thus may be obtained in a high yield. Therefore, the novel shTACE has a cost advantage compared with the conventional gene construct, which is limited to use of Maxiprep and requires repeated Prep (e.g., Miniprep) due to low yield. In addition, since endotoxins are automatically removed during a Maxiprep experiment, the prepared shTACE may be injected into the body immediately after obtaining the shTACE. The novel shTACE of the present invention is represented by SEQ ID NO: 1.

Since a gene has a negative charge due to phosphates thereof, it cannot easily penetrate the cell membrane exhibiting a negative charge due to electrical repulsion between the gene and the cell membrane. Therefore, when a gene reacts with a substance exhibiting a positive charge to form a complex in which a net charge is positive, the complex including the gene may enter the cell more easily, and as a result, the gene may be expressed within the cell. Such a substance that enhances gene delivery into cells is called a gene carrier. The gene carrier refers to a substance that binds to a gene and promotes gene delivery to enhance intracellular expression of the gene. Such gene carriers are mostly positively charged, and a gene/carrier complex is formed by electrical interaction between a negatively charged gene and a positively charged gene carrier.

According to one embodiment of the present invention, the low stability of shTACE may be overcome by using a nonviral gene carrier (hereinafter, referred to as “PAs-s”) including the acetate of disulfide-linked poly(oligo-arginine).

The disulfide-linked poly(oligo-arginine) may be composed of nine-arginine oligomers, wherein each nine-arginine oligomer includes disulfide-linked cysteines at both ends thereof.

The disulfide-linked poly(oligo-arginine) may include a repeating unit of Cys-(9×Arg)-Cys, and the thiol groups of cysteines at both ends of the repeating unit may be polymerized via disulfide crosslinking.

First, a monomeric peptide of Cys-(9×Arg)-Cys may be synthesized using Fmoc solid-phase peptide synthesis, in which each amino acid is extended one by one according to a predetermined sequence order and the α-amino group of the amino acid is protected with a 9-fluorenyl-methyloxycarbonyl (Fmoc) group. When a step of elongating the peptide chain is completed, a released form of the peptide is obtained by treatment with trifluoroacetic acid (TFA). Subsequently, a monomeric peptide of Cys-(9×Arg)-Cys in a TFA salt form is converted into an acetate form. That is, the TFA salt is substituted with acetate using ion exchange chromatography with AG1-X8 resins.

The monomeric peptide of Cys-(9×Arg)-Cys in an acetate form is subjected to an oxidative polymerization reaction, in which disulfide bonds between cysteines are formed, and as a result, a polymeric gene carrier is formed.

In the case of PAs-s used as a gene carrier in the present invention, the basic salt form of the gene carrier is changed from a TFA salt to an acetate form, unlike conventional gene carriers. This may minimize side effects that may occur when the gene carrier is injected into the body, and may further improve the ability of the gene carrier to carry a gene in the body compared to conventional gene carriers.

The shTACE represented by SEQ ID NO: 1 and PAs-s form a complex via electrical interaction. To form a gene/carrier complex (peptoplex) with an excellent therapeutic effect, the ratio of a gene to a gene carrier should be optimally adjusted when the complex is formed. There are various kinds of ratios such as weight ratio, charge ratio, and nitrogen/phosphorous (N/P) ratio. In the present invention, a weight ratio is used. In the gene/carrier complex according to the present invention, TACE shRNA and the gene carrier are preferably mixed in a weight ratio of 1:1.5 to 8 or 1:2 to 4.

In addition, according to one embodiment of the present invention, the low stability of shTACE may be overcome by using a nonviral gene carrier (hereinafter, referred to as PDPR) including the trifluoroacetic acid (TFA) salt of poly(oligo-aspartic acid)poly(oligo-arginine).

The poly(oligo-aspartic acid)poly(oligo-arginine) may include cysteines at both ends thereof.

The poly(oligo-aspartic acid)poly(oligo-arginine) may include cysteines at both ends thereof and may be composed of an eight-aspartic acid oligomer and a sixteen-arginine oligomer.

The poly(oligo-aspartic acid)poly(oligo-arginine) may refer to a Cys-(8×Asp)-(16×Arg)-Cys peptide.

First, a Cys-(8×Asp)-(16×Arg)-Cys peptide may be synthesized using Fmoc solid-phase peptide synthesis, in which each amino acid is extended one by one according to a predetermined sequence order and the α-amino group of the amino acid is protected with a 9-fluorenyl-methyloxycarbonyl (Fmoc) group. When a step of elongating the peptide chain is completed, a released form of the peptide is obtained by treatment with trifluoroacetic acid (TFA).

The TFA salt of Cys-(8×Asp)-(16×Arg)-Cys (hereinafter, referred to as 8D16R) according to the present invention may form a gene carrier.

Unlike conventional gene carriers, the PDPR gene carrier used in the present invention is characterized in that the PDPR gene carrier is composed of a peptide targeting a bone resorption site and a peptide capable of increasing the intracellular delivery efficiency of the peptide. This may minimize side effects that may occur when the gene carrier is injected into the body, and may further improve the ability of the gene carrier to carry a gene in the body compared to conventional gene carriers.

The shTACE represented by SEQ ID NO: 1 and PDPR form a complex via electrical interaction. To form a gene/carrier complex (peptoplex) with an excellent therapeutic effect, the ratio of a gene to a gene carrier should be optimally adjusted when the complex is formed. There are various kinds of ratios such as weight ratio, charge ratio, and nitrogen/phosphorous (N/P) ratio. In the present invention, weight ratio is used. In the gene/carrier complex according to the present invention, TACE shRNA and the gene carrier are preferably mixed in a weight ratio of 1:1.5 to 8 or 1:2 to 6.

In addition, the present invention provides a method of preparing a gene/carrier complex, the method including a step of mixing and incubating tumor necrosis factor-α converting enzyme (TNF-α converting enzyme, TACE) shRNA represented by SEQ ID NO: 1 and a nonviral carrier.

To obtain an optimal gene/carrier complex, incubation is preferably performed at 20 to 40° C. for 20 to 40 minutes. At higher temperatures above 40° C., hydrogen bonds between strands of DNA break, resulting in DNA denaturation. Since a carrier is produced by polymerization of peptides and is therefore denatured at high temperature, the incubation is preferably performed at a temperature of 40° C. or below. Furthermore, cells are preferably treated with the complex at a temperature similar to a body temperature. In addition, when the incubation is performed for more than 40 minutes, the gene and the gene carrier form a precipitate, so that it is preferable that the incubation time not exceed 40 minutes. Also, after the gene and the carrier form a complex via electrical attraction, it is preferable to incubate the complex for at least 20 minutes to maintain the state of the complex stably.

TACE short hairpin RNA (TACE shRNA or shTACE, SEQ ID NO: 1) exhibits a negative charge, and PAs-s or PDPR exhibits a positive charge. When the two components are mixed and incubated at room temperature for about 20 to 40 minutes, a gene/carrier complex may be formed via electrostatic attraction. After complex formation, the final volume is adjusted using deionized/distilled water, PBS, and the like for each group.

To determine the optimal weight ratio of a gene/carrier complex, the concentrations of a gene and a gene carrier should first be determined, respectively. Since the amount of ultraviolet radiation absorbed is proportional to the amount of DNA, the concentration of the gene is measured using an ultraviolet spectrophotometer. It is preferable to prepare the gene at a concentration of 1 mg/ml or less to prevent precipitation of the gene/carrier complex. The gene carrier may be synthesized at a final concentration of 1 mg/ml by adjusting the amount of HEPES buffer.

Any of the above-described contents relating to the gene/carrier complex may be directly applied to the method of preparing the gene/carrier complex.

In addition, the present invention provides a composition for preventing or treating inflammatory diseases, including the gene/carrier complex as an active ingredient.

As used herein, inflammatory diseases may include one or more selected from the group consisting of ocular inflammation, allergic conjunctivitis, dermatitis, rhinitis, asthma, rheumatoid arthritis, acute lung injury, inflammatory bowel disease, and obesity.

As used herein, the term “ocular inflammation” may include, for example, iritis, uveitis, episcleritis, scleritis, keratitis, endophthalmitis, blepharitis, and iatrogenic inflammatory conditions.

As used herein, the term “allergic conjunctivitis” refers to inflammation of the conjunctiva located in the eyelid and covering the exposed surface of the sclera. “Allergic conjunctivitis” may include, for example, atopic keratoconjunctivitis, giant papillary conjunctivitis, hay fever conjunctivitis, perennial allergic conjunctivitis, and vernal keratoconjunctivitis.

As used herein, the term “dermatitis” refers to inflammation of the skin and may include, for example, allergic contact dermatitis, hives, non-sebaceous dermatitis (dry skin of legs), atopic dermatitis, contact dermatitis (e.g., irritant contact dermatitis and lacquer-induced contact dermatitis), eczema, gravitational dermatitis, nummular dermatitis, otitis externa, perioral dermatitis, and seborrheic dermatitis.

As used herein, the term “rhinitis” refers to inflammation of the nasal mucosa and may include, for example, allergic rhinitis, atopic rhinitis, irritant rhinitis, acidophilic non-allergic rhinitis, medicamentous rhinitis, and neutrophilic rhinosinusitis.

As used herein, the term “asthma” refers to inflammation of the respiratory tract causing stenosis of the airways moving air from the nose and mouth to the lungs and may include, for example, allergic asthma, atopic asthma, atopic bronchus IgE-mediated asthma, bronchus asthma, bronchiolitis, emphysematous asthma, essential asthma, exercise-induced asthma, exogenous asthma induced by environmental factors, incipient asthma, endogenous asthma caused by pathophysiological disorders, non-allergic asthma, non-atopic asthma, and wheezy infant syndrome.

As used herein, the term “rheumatoid arthritis” is an inflammatory systemic autoimmune disease. When the disease occurs, the synovial membrane is mainly attacked by the autoimmune system. The cause of the disease is unknown, and the symptoms thereof include polyarthritis and chronic inflammation in various tissues and organs. Thus, rheumatoid arthritis is also a type of chronic inflammatory disease.

As used herein, the term “acute lung injury” refers to damage due to acute inflammation of the lungs, where the epithelial and endothelial cells of the lungs are damaged. Symptoms include respiratory failure and arterial hypoxemia.

As used herein, the term “obesity” refers to a state of excessive fat tissue in the body. Obesity is a low-stage inflammatory disease and may cause metabolic syndrome such as type 2 diabetes.

As used herein, the term “inflammatory bowel disease” refers to severe chronic inflammation caused by inflammatory agents (e.g., inflammatory cytokines) in the gastrointestinal tract, and specific examples thereof may include Crohn's disease, ulcerative colitis, intestinal Behcet's disease, simple ulcers, radiation enteritis, and ischemic colitis.

The pharmaceutical composition of the present invention may be administered together with a pharmaceutically acceptable carrier. Upon oral administration, the pharmaceutical composition may further contain, in addition to an active ingredient, a binder, a lubricant, a disintegrant, an excipient, a solubilizer, a dispersant, a stabilizer, a suspending agent, a pigment, a perfume, and the like. When the pharmaceutical composition of the present invention is injected, the pharmaceutical composition may be mixed with a buffer, a preservative, a pain relief agent, a solubilizer, an isotonic agent, a stabilizer, and the like. Upon topical administration, the composition of the present invention may include a base, an excipient, a lubricant, a preservative, and the like.

Formulations of the composition of the present invention may be prepared in a variety of ways by mixing with pharmaceutically acceptable carriers as described above. For example, upon oral administration, the composition of the present invention may be prepared in the form of tablets, troches, capsules, elixirs, suspensions, syrups, wafers, and the like. When the composition of the present invention is injected, the composition may be prepared in unit dose ampoules or in multiple unit dose forms. The composition of the present invention may be formulated into other solutions, suspensions, tablets, pills, capsules, sustained release formulations, and the like.

In addition, examples of suitable carriers, excipients, and diluents for formulation may include lactose, dextrose, sucrose, sorbitol, mannitol, xylitol, erythritol, maltitol, starch, acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, and the like. In addition, fillers, anti-coagulants, lubricants, wetting agents, perfumes, preservatives, and the like may be further included in the formulation

The pharmaceutical composition of the present invention may be administered orally or parenterally. For examples, the pharmaceutical composition may be administered through oral, aerosol, buccal, epidermal, intradermal, inhalation, intramuscular, intranasal, intraocular, intrapulmonary, intravenous, intraperitoneal, nasal, ocular, oral, ear, injection, patch, subcutaneous, hypoglossal, topical or percutaneous routes, without being limited thereto.

For clinical administration, the pharmaceutical composition of the present invention may be formulated into a suitable formulation using known techniques. For example, upon oral administration, the composition may be mixed with an inert diluent or edible carrier, sealed in a hard or soft gelatin capsule, or tableted. For oral administration, the active ingredient of the composition may be mixed with an excipient and used in the form of ingestible tablets, buccal tablets, troches, capsules, elixirs, suspensions, syrups, wafers, and the like. In addition, various formulations for injection, parenteral administration, and the like may be prepared according to known techniques or conventional techniques in the art.

The effective dose of the pharmaceutical composition of the present invention may be determined depending on the patient's body weight, age, sex, health conditions, diet, administration time, administration method, excretion rate, disease severity, and the like, and may be easily determined by an ordinary export in the art.

The preferred dosage of the pharmaceutical composition of the present invention may vary depending on the condition and weight of the patient, the degree of disease, the type of drug, the route of administration, and the duration of administration, and may be appropriately selected by those skilled in the art. Preferably, the composition is administered at a daily dose of 0.001 to 100 mg/kg body weight, more preferably 0.01 to 30 mg/kg body weight. Administration may be carried out once or several times a day. The gene carrier complex of the present invention may be present in an amount of 0.0001 to 10% by weight, preferably 0.001 to 1% by weight, with respect to 100% by weight of the total composition.

The pharmaceutical composition of the present invention may be administered to mammals such as rats, mice, livestock, and humans via various routes. There are no limitations on the method of administration, and for example, the composition may be administered orally or rectally, or by intravenous, intramuscular, subcutaneous, intrauterine dural or intracerebroventricular injection.

Hereinafter, the present invention will be described in detail with reference to examples. However, the following examples are illustrative of the present invention, and the present invention is not limited to the following examples.

### EXAMPLES

### Preparation Example 1: Preparation of shTACE

shTACE plasmid DNA consists of a total 6,669 base pairs and contains a U6 promoter. The plasmid vector was constructed to selectively down-regulate the expression of TACE by inserting a DNA sequence (ACACCTGCTGCAATAGTGA) consisting of 19 bases. An SV40 ori and a pUC ori were used for the proliferation and expression of the vector. An ampicillin resistance gene was inserted into the vector. To select cells stably expressing shTACE among cells transfected with the vector, a puromycin resistance gene was introduced into the vector as a selection marker. An eGFP reporter gene was introduced into the vector to determine whether the vector was correctly inserted. The sequence of a hairpin loop in the vector is TCAAGAG. The sequence of the shTACE plasmid prepared by the above method is as follows and is represented by SEQ ID NO: 1.

GAATTCGCGGCCCTAGCTTGGGATCTTTGTGAAGGAACCTTACTTCTGT
GGTGTGACATAATTGGACAAACTACCTACAGAGATTTAAAGCTCTAAGG
TAAATATAAAATTTTTAAGTGTATAATGTGTTAAACTAGCTGCATATGC
TTGCTGCTTGAGAGTTTTGCTTACTGAGTATGATTTATGAAAATATTAT
ACACAGGAGCTAGTGATTCTAATTGTTTGTGTATTTTAGATTCACAGTC
CCAAGGCTCATTTCAGGCCCCTCAGTCCTCACAGTCTGTTCATGATCAT
AATCAGCCATACCACATTTGTAGAGGTTTTACTTGCTTTAAAAAACCTC
CCACACCTCCCCCTGAACCTGAAACATAAAATGAATGCAATTGTTGTTG
TTAACTTGTTTATTGCAGCTTATAATGGTTACAAATAAAGCAATAGCAT
CACAAATTTCACAAATAAAGCATTTTTTTCACTGCATTCTAGTTGTGGT
TTGTCCAAACTCATCAATGTATCTTATCATGTCTGGATCGATCCTGCAT
TAATGAATCGGCCAACGCGCGGGGAGAGGCGGTTTGCGTATTGGCTGGC
GTAATAGCGAAGAGGCCCGCACCGATCGCCCTTCCCAACAGTTGCGCAG
CCTGAATGGCGAATGGGACGCGCCCTGTAGCGGCGCATTAAGCGCGGCG
GGTGTGGTGGTTACGCGCAGCGTGACCGCTACACTTGCCAGCGCCCTAG
CGCCCGCTCCTTTCGCTTTCTTCCCTTCCTTTCTCGCCACGTTCGCCGG
CTTTCCCCGTCAAGCTCTAAATCGGGGGCTCCCTTTAGGGTTCCGATTT
AGTGCTTTACGGCACCTCGACCCCAAAAAACTTGATTAGGGTGATGGTT
CACGTAGTGGGCCATCGCCCTGATAGACGGTTTTTCGCCCTTTGACGTT
GGAGTCCACGTTCTTTAATAGTGGACTCTTGTTCCAAACTGGAACAACA
CTCAACCCTATCTCGGTCTATTCTTTTGATTTATAAGGGATTTTGCCGA
TTTCGGCCTATTGGTTAAAAAATGAGCTGATTTAACAAATATTTAACGC
GAATTTTAACAAAATATTAACGTTTACAATTTCGCCTGATGCGGTATTT
TCTCCTTACGCATCTGTGCGGTATTTCACACCGCATACGCGGATCTGCG
CAGCACCATGGCCTGAAATAACCTCTGAAAGAGGAACTTGGTTAGGAAC
CTTCTGAGGCGGAAAGAACCAGCTGTGGAATGTGTGTCAGTTAGGGTGT
GGAAAGTCCCCAGGCTCCCCAGCAGGCAGAAGTATGCAAAGCATGCATC
TCAATTAGTCAGCAACCAGGTGTGGAAAGTCCCCAGGCTCCCCAGCAGG
CAGAAGTATGCAAAGCATGCATCTCAATTAGTCAGCAACCATAGTCCCG
CCCCTAACTCCGCCCATCCCGCCCCTAACTCCGCCCAGTTCCGCCCATT
CTCCGCCCCATGGCTGACTAATTTTTTTTATTTATGCAGAGGCCGAGGC
CGCCTCGGCCTCTGAGCTATTCCAGAAGTAGTGAGGAGGCTTTTTTGGA
GGCCTAGGCTTTTGCAAAAAGCTTGATTCTTCTGACACAACAGTCTCGA
ACTTAAGGCTAGAGCCACCATGACCGAGTACAAGCCCACGGTGCGCCTC
GCCACCCGCGACGACGTCCCCAGGGCCGTACGCACCCTCGCCGCCGCGT
TCGCCGACTACCCCGCCACGCGCCACACCGTCGATCCGGACCGCCACAT
CGAGCGGGTCACCGAGCTGCAAGAACTCTTCCTCACGCGCGTCGGGCTC
GACATCGGCAAGGTGTGGGTCGCGGACGACGGCGCCGCGGTGGCGGTCT
GGACCACGCCGGAGAGCGTCGAAGCGGGGGCGGTGTTCGCCGAGATCGG
CCCGCGCATGGCCGAGTTGAGCGGTTCCCGGCTGGCCGCGCAGCAACAG
ATGGAAGGCCTCCTGGCGCCGCACCGGCCCAAGGAGCCCGCGTGGTTCC
TGGCCACCGTCGGCGTCTCGCCCGACCACCAGGGCAAGGGTCTGGGCAG
CGCCGTCGTGCTCCCCGGAGTGGAGGCGGCCGAGCGCGCCGGGGTGCCC
GCCTTCCTGGAGACCTCCGCGCCCCGCAACCTCCCCTTCTACGAGCGGC
TCGGCTTCACCGTCACCGCCGACGTCGAGGTGCCCGAAGGACCGCGAAC
CTGGTGCATGACCCGCAAGCCCGGTGCCTGAGTTTAACGAAATGACCGA
CCAAGCGACGCCCAACCTGCCATCACGATGGCCGCAATAAAATATCTTT
ATTTTCATTACATCTGTGTGTTGGTTTTTTGTGTGGATCGATAGCGATA
AGGATCGATCCGCGCATGGTGCACTCTCAGTACAATCTGCTCTGATGCC
GCATAGTTAAGCCAGCCCCGACACCCGCCAACACCCGCTGACGCGCCCT
GACGGGCTTGTCTGCTCCCGGCATCCGCTTACAGACAAGCTGTGACCGT
CTCCGGGAGCTGCATGTGTCAGAGGTTTTCACCGTCATCACCGAAACGC
GCGAGACGAAAGGGCCTCGTGATACGCCTATTTTTATAGGTTAATGTCA
TGATAATAATGGTTTCTTAGACGTCAGGTGGCACTTTTCGGGGAAATGT
GCGCGGAACCCCTATTTGTTTATTTTTCTAAATACATTCAAATATGTAT
CCGCTCATGAGACAATAACCCTGATAAATGCTTCAATAATATTGAAAAA
GGAAGAGTATGAGTATTCAACATTTCCGTGTCGCCCTTATTCCCTTTTT
TGCGGCATTTTGCCTTCCTGTTTTTGCTCACCCAGAAACGCTGGTGAAA
GTAAAAGATGCTGAAGATCAGTTGGGTGCACGAGTGGGTTACATCGAAC
TGGATCTCAACAGCGGTAAGATCCTTGAGAGTTTTCGCCCCGAAGACCG
TTTTCCAATGATGAGCACTTTTAAAGTTCTGCTATGTGGCGCGGTATTA
TCCCGTATTGACGCCGGGCAAGAGCAACTCGGTCGCCGCATACACTATT
CTCAGAATGACTTGGTTGAGTACTCACCAGTCACAGAAAAGCATCTTAC
GGATGGCATGACAGTAAGAGAATTATGCAGTGCTGCCATAACCATGAGT
GATAACACTGCGGCCAACTTACTTCTGACAACGATCGGAGGACCGAAGG
AGCTAACCGCTTTTTTGCACAACATGGGGGATCATGTAACTCGCCTTGA
TCGTTGGGAACCGGAGCTGAATGAAGCCATACCAAACGACGAGCGTGAC
ACCACGATGCCTGTAGCAATGGCAACAACGTTGCGCAAACTATTAACTG
GCGAACTACTTACTCTAGCTTCCCGGCAACAATTAATAGACTGGATGGA
GGCGGATAAAGTTGCAGGACCACTTCTGCGCTCGGCCCTTCCGGCTGGC
TGGTTTATTGCTGATAAATCTGGAGCCGGTGAGCGTGGGTCTCGCGGTA
TCATTGCAGCACTGGGGCCAGATGGTAAGCCCTCCCGTATCGTAGTTAT
CTACACGACGGGGAGTCAGGCAACTATGGATGAACGAAATAGACAGATC
GCTGAGATAGGTGCCTCACTGATTAAGCATTGGTAACTGTCAGACCAAG
TTTACTCATATATACTTTAGATTGATTTAAAACTTCATTTTTAATTTAA
AAGGATCTAGGTGAAGATCCTTTTTGATAATCTCATGACCAAAATCCCT
TAACGTGAGTTTTCGTTCCACTGAGCGTCAGACCCCGTAGAAAAGATCA
AAGGATCTTCTTGAGATCCTTTTTTTCTGCGCGTAATCTGCTGCTTGCA
AACAAAAAAACCACCGCTACCAGCGGTGGTTTGTTTGCCGGATCAAGAG
CTACCAACTCTTTTTCCGAAGGTAACTGGCTTCAGCAGAGCGCAGATAC
CAAATACTGTCCTTCTAGTGTAGCCGTAGTTAGGCCACCACTTCAAGAA
CTCTGTAGCACCGCCTACATACCTCGCTCTGCTAATCCTGTTACCAGTG
GCTGCTGCCAGTGGCGATAAGTCGTGTCTTACCGGGTTGGACTCAAGAC
GATAGTTACCGGATAAGGCGCAGCGGTCGGGCTGAACGGGGGGTTCGTG
CACACAGCCCAGCTTGGAGCGAACGACCTACACCGAACTGAGATACCTA
CAGCGTGAGCATTGAGAAAGCGCCACGCTTCCCGAAGGGAGAAAGGCGG
ACAGGTATCCGGTAAGCGGCAGGGTCGGAACAGGAGAGCGCACGAGGGA
GCTTCCAGGGGGAAACGCCTGGTATCTTTATAGTCCTGTCGGGTTTCGC
CACCTCTGACTTGAGCGTCGATTTTTGTGATGCTCGTCAGGGGGGCGGA
GCCTATGGAAAAACGCCAGCAACGCGGCCTTTTTACGGTTCCTGGCCTT
TTGCTGGCCTTTTGCTCACATGTTCTTTCCTGCGTTATCCCCTGATTCT
GTGGATAACCGTATTACCGCCTTTGAGTGAGCTGATACCGCTCGCCGCA
GCCGAACGACCGAGCGCAGCGAGTCAGTGAGCGAGGAAGCGGAAGAGCG
CCCAATACGCAAACCGCCTCTCCCCGCGCGTTGGCCGATTCATTAATGC
AGAGCTTGCAATTCGCGCGTTTTTCAATATTATTGAAGCATTTATCAGG
GTTATTGTCTCATGAGCGGATACATATTTGAATGTATTTAGAAAAATAA
ACAAATAGGGGTTCCGCGCACATTTCCCCGAAAAGTGCCACCTGACGTC
TAAGAAACCATTATTATCATGACATTAACCTATAAAAATAGGCGTAGTA
CGAGGCCCTTTCACTCATTAGATGCATGTCGTTACATAACTTACGGTAA
ATGGCCCGCCTGGCTGACCGCCCAACGACCCCCGCCCATTGACGTCAAT
AATGACGTATGTTCCCATAGTAACGCCAATAGGGACTTTCCATTGACGT
CAATGGGTGGAGTATTTACGGTAAACTGCCCACTTGGCAGTACATCAAG
TGTATCATATGCCAAGTACGCCCCCTATTGACGTCAATGACGGTAAATG
GCCCGCCTGGCATTATGCCCAGTACATGACCTTATGGGACTTTCCTACT
TGGCAGTACATCTACGTATTAGTCATCGCTATTACCATGGTGATGCGGT
TTTGGCAGTACATCAATGGGCGTGGATAGCGGTTTGACTCACGGGGATT
TCCAAGTCTCCACCCCATTGACGTCAATGGGAGTTTGTTTTGGCACCAA
AATCAACGGGACTTTCCAAAATGTCGTAACAACTCCGCCCCATTGACGC
AAATGGGCGGTAGGCGTGTACGGTGGGAGGTCTATATAAGCAGAGCTCG
TTTAGTGAACCGTCAGATCGCCTGGAGACGCCATCCACGCTGTTTTGAC
CTCCATAGAAGACACCGGGACCGATCCAGCCTCCGGACTCTAGCCTAGG
CCGCGGACCATGTCCGGCTTGAACGACATCTTCGAGGCCCAGAAGATCG
AGTGGCACGAGGAAAAGCTTCGAACCATGGTGAGCAAGGGCGAGGAGCT
GTTCACCGGGGTGGTGCCCATCCTGGTCGAGCTGGACGGCGACGTAAAC
GGCCACAAGTTCAGCGTGTCCGGCGAGGGCGAGGGCGATGCCACCTACG
GCAAGCTGACCCTGAAGTTCATCTGCACCACCGGCAAGCTGCCCGTGCC
CTGGCCCACCCTCGTGACCACCCTGACCTACGGCGTGCAGTGCTTCAGC
CGCTACCCCGACCACATGAAGCAGCACGACTTCTTCAAGTCCGCCATGC
CCGAAGGCTACGTCCAGGAGCGCACCATCTTCTTCAAGGACGACGGCAA
CTACAAGACCCGCGCCGAGGTGAAGTTCGAGGGCGACACCCTGGTGAAC
CGCATCGAGCTGAAGGGCATCGACTTCAAGGAGGACGGCAACATCCTGG
GGCACAAGCTGGAGTACAACTACAACAGCCACAACGTCTATATCATGGC
CGACAAGCAGAAGAACGGCATCAAGGTGAACTTCAAGATCCGCCACAAC
ATCGAGGACGGCAGCGTGCAGCTCGCCGACCACTACCAGCAGAACACCC
CCATCGGCGACGGCCCCGTGCTGCTGCCCGACAACCACTACCTGAGCAC
CCAGTCCGCCCTGAGCAAAGACCCCAACGAGAAGCGCGATCACATGGTC
CTGCTGGAGTTCGTGACCGCCGCCGGGATCACTCTCGGCATGGACGAGC
TGTACAAGTAGCTCGAGTGCGGCCCCAAATAATGATTTTATTTTGACTG
ATAGTGACCTGTTCGTTGCAACAAATTGATGAGCAATGCTTTTTTATAA
TGCCAACTTTGTACAAAAAAGCAGGCTGCGATCGCTCGGGCAGGAAGAG
GGCCTATTTCCCATGATTCCTTCATATTTGCATATACGATACAAGGCTG
TTAGAGAGATAATTAGAATTAATTTGACTGTAAACACAAAGATATTAGT
ACAAAATACGTGACGTAGAAAGTAATAATTTCTTGGGTAGTTTGCAGTT
TTAAAATTATGTTTTAAAATGGACTATCATATGCTTACCGTAACTTGAA
AGTATTTCGATTTCTTGGGTTTATATATCTTGTGGAAAGGACGAGgatc
cgacacctgctgcaatagtgatcaagagtcactattgcagcaggtgttt
ttttg

### Preparation Example 2: Preparation of Gene Carrier (PAs-s)

A gene carrier (PAs-s) including the acetate of disulfide-linked poly(oligo-arginine) is formed by polymerizing a monomeric peptide of Cys-(9×Arg)-Cys represented by SEQ ID NO: 4, in which a nine-arginine oligomer including cysteines at both ends thereof is a basic repeating unit.

First, a monomeric peptide of Cys-(9×Arg)-Cys was synthesized using Fmoc solid-phase peptide synthesis, in which each amino acid was extended one by one according to a predetermined sequence order and the α-amino group of the amino acid was protected with a 9-fluorenyl-methyloxycarbonyl (Fmoc) group. When a step of elongating the peptide chain was completed, a released form of the peptide was obtained by treatment with trifluoroacetic acid (TFA). Subsequently, a monomeric peptide of Cys-(9×Arg)-Cys in a TFA salt form was converted into an acetate form. That is, the TFA salt was substituted with acetate using ion exchange chromatography with AG1-X8 resins.

A monomeric peptide of Cys-(9×Arg)-Cys in an acetate form was subjected to oxidative polycondensation, in which disulfide bonds between cysteines were formed, and as a result, the acetate of disulfide-linked poly(oligo-arginine), a polymeric carrier, was formed. For the oxidative polymerization reaction, the peptide of Cys-(9×Arg)-Cys was added to PBS containing 30% dimethyl sulfoxide (DMSO) and stirred for 6 days at a rate of 150 rpm. After stirring, the reaction was terminated by adding a HEPES buffer at a concentration of 5 mmol/L.

### Preparation Example 3: Preparation of Gene Carrier (8D16R)

Preparation of a gene carrier (8D16R) was commissioned by Peptron Company (Daejeon, Korea). The gene carrier including the TFA salt of poly(oligo-aspartic acid)poly(oligo-arginine) includes a peptide of Cys-(8×Asp)-(16×Arg)-Cys represented by SEQ ID NO: 5, which is composed of eight aspartic acids and sixteen arginines and has cysteines at both ends thereof.

First, a peptide of Cys-(8×Asp)-(16×Arg)-Cys was synthesized using Fmoc solid-phase peptide synthesis, in which each amino acid was extended one by one according to a predetermined sequence order and the α-amino group of the amino acid was protected with a 9-fluorenyl-methyloxycarbonyl (Fmoc) group. When a step of elongating the peptide chain was completed, a released form of the peptide (8D16R) was obtained by treatment with trifluoroacetic acid (TFA).

When a step of elongating the peptide chain was completed, the Cys-(8×Asp)-(16×Arg)-Cys peptide was weighed, and the concentration thereof was adjusted with deionized/distilled water.

### Experimental Example 1: Preparation of shTACE/PAs-s Complex and Evaluation of Effectiveness Thereof

### Experimental Procedure

<Cell Culture>

Dulbecco's Modified Eagle medium (DMEM) and fetal bovine serum (FBS) were purchased from WelGENE Inc. (Korea). RAW 264.7 cells, mouse-derived macrophages, were purchased from the Korean Cell Line Bank and subcultured once every two days. Cells were cultured in complete medium supplemented with 10% FBS, penicillin (100 IU/ml), and streptomycin (100 μg/ml) at 37° C. and 5% CO2 atmospheric conditions.

<In Vitro Cytotoxicity>

Cell viability was measured using 3-(4,5-dimethylthiazol-2-yl)-2,5-diphenyltetrazolium bromide (MTT) assay. Mouse-derived macrophages (RAW 264.7 cells) were cultured in a cell culture plate at a density of 4×10⁴ cells per 1 ml of complete medium (CM). 24 hours after cell culture, the cells were treated with a gene/carrier complex. At this time, the amount or the weight ratio of the complex was adjusted according to each experimental purpose. After 24 hours, the cells were treated with 5 mg/ml of MTT and incubated at 37° C. for 4 hours. Thereafter, the complete medium was removed from the cells, and DMSO of the same amount as the amount of the complete medium was added to the cells, followed by incubation for 15 minutes. After incubation, the cell culture plate was placed in an absorbance measurement apparatus, and absorbance was measured at a wavelength of 570 nm. Based on the obtained results, relative cell viability was calculated. The principle of MTT assay is to take advantage of the ability of mitochondria to reduce MTT, a water-soluble yellow tetrazole, to a water-insoluble purple formazan(3-(4,5-dimethylthiazol-2-yl)-2, 5-diphenyltetrazolium bromide) product using NADPH, NADH, and the like. Since MTT formazan is insoluble in water, MTT formazan is dissolved in DMSO, an organic solvent, and absorbance is measured at 570 nm. At this time, the absorbance is proportional to the metabolic activity of the cells. Based on the measured absorbance, relative cell viability may be evaluated.

<In Vitro Transfection Efficiency>

The transfection efficiency of a PAs-s carrier was measured using luciferase assay. Luciferase enzyme is expressed from a luciferase plasmid (pLuci) in cells. The enzymatic activity of luciferase oxidizes luciferin to emit light energy, and thus pLuci acts as a reporter gene that indirectly indicates the expression level of a foreign gene introduced into cells. Each well of a cell culture plate was inoculated with 4×10⁴ mouse-derived macrophages (RAW 264.7 cells) and cultured for 24 hours. After culture, 100 ng/ml of lipopolysaccharide (LPS) was added to each well to activate macrophages. Activated or unactivated macrophages were treated with a pLuci/PAs-s complex formed by mixing the luciferase plasmid (pLuci) and the PAs-s carrier. The gene/carrier complex was prepared in a plain medium (PM). The optimal weight ratio of the gene (pLuci) to the PAs-s carrier was 1:2. In this case, the amount of the gene used was 1 μg. Accordingly, the amount of the PAs-s carrier used was 2 μg. Thus, cells were treated with the pLuci/PAs-s complex formed using these amounts. A PEI carrier was used as a control.

After 48 hours, 200 μl of 1× reporter lysis buffer was added to each well, followed by incubation at 4° C. for 15 minutes. Then, cells in each well were collected and lysed. Then, the cell lysates were subjected to centrifugation at 1,2470 g for 3 minutes to separate cell culture supernatants, and the cell supernatants were mixed with a luciferase assay reagent containing a luciferase substrate. The light emitted by chemical reaction between the cell lysates and the luciferase substrate was detected using a luminometer (Berthold Detection Systems) and expressed as a relative luminescence unit (RLU). The obtained results were expressed as RLU/mg of cell protein. Protein concentration was calculated using a DC protein assay kit including a bovine serum albumin standard. A luciferase analysis kit was purchased from Promega (USA). In other words, the transfection efficiency of the PAs-s carrier was determined by the degree of chemical reaction between a luciferase substrate and cell lysates.

<RNA Isolation and Verification of TACE mRNA Knockdown>

Each well of a cell culture plate was inoculated with 4×10⁴ mouse-derived macrophages (RAW 264.7 cells) and cultured for 24 hours. After culture, 100 ng/ml of lipopolysaccharide (LPS) was added to each well to activate macrophages. Activated or unactivated macrophages were treated with a gene/carrier complex (shTACE/PAs-s peptoplex) and incubated for 24 hours. A PEI complex was used as a control. After incubation, cells were collected from each well, and the collected cells were centrifuged at 10,000 rpm to obtain cell pellets. Then, the cell lysates were homogenized and RNA was isolated using an RNeasy Lipid Tissue Mini Kit (Qiagen). For each group, 1 μg of RNA was reacted with reverse transcriptase to synthesize cDNA complementary to RNA of each group. Then, RT-PCR was performed on cDNA using a Cyber Premix Ex Taq RT-PCR kit, and the relative mRNA level of TACE was calculated based on the mRNA level of GAPDH, an endogenous control. At this time, a forward primer and a reverse primer for TACE amplification were 5′-GTACGTCGATGCAGAGCAAA-3′ (SEQ ID NO: 2) and 5′-AAACCAGAACAGACCCAACG-3′ (SEQ ID NO: 3), respectively.

<Measurement of TNF-α Level>

Each well of a 12-well cell culture plate was inoculated with 4×10⁴ mouse-derived macrophages (RAW 264.7 cells) and cultured for 24 hours. After culture, 100 ng/ml of lipopolysaccharide (LPS) was added to each well to activate macrophages. Activated or unactivated macrophages were treated with a gene/carrier complex (shTACE/PAs-s peptoplex) or tumor necrosis factor-α (TNF-α)-targeting monoclonal antibodies (Infliximab). After 48 hours of incubation, 1 ml of cell culture fluid was obtained from each well. The cell culture fluid was subjected to centrifugation at 4° C. and 13,000 rpm for 5 minutes to separate a supernatant. Then, for each group, the amount of TNF-α, a cytokine present in the supernatant was measured using Sandwich ELISA (eBioscience). The relative amount of TNF-α was obtained by dividing the amount of TNF-α by the amount of cellular protein. The obtained results were expressed as the amount of TNF-α per mg of cellular protein.

<Animal Modeling for Ulcerative Colitis and Injection of Therapeutic Gene Complex>

Animal modeling for ulcerative colitis was performed by supplying an aqueous dextran sodium sulfate (DSS) solution to C57BL/6 mice instead of ordinary water. A gene/carrier complex (shTACE/PAs-s peptoplex) incubated for 30 minutes was intravenously injected before the onset of ulcerative colitis modeling or after completion of the modeling.

In the case of experiments conducted for the purpose of disease prevention, acute ulcerative colitis modeling was performed by supplying water containing 5% DSS to mice. Before starting modeling, a gene/carrier complex (shTACE/PAs-s peptoplex) formed by mixing 20 μg of the gene and 40 μg of the gene carrier (i.e., mixed in an optimal weight ratio of 1:2) was intravenously injected for two consecutive days. At this time, the amount of the complex injected to the experimental animals was about 1 mg/kg.

In the case of experiments conducted for the purpose of disease treatment, chronic ulcerative colitis modeling was performed by supplying water containing 3% DSS to mice. A gene/carrier complex (shTACE/PAs-s peptoplex) formed by mixing 20 μg of the gene and 40 μg of the gene carrier (i.e., mixed in an optimal weight ratio of 1:2) was intravenously injected for three consecutive days. At this time, the amount of the complex injected to the experimental animals was about 1 mg/kg. In addition, a short hairpin RNA (shNS)/PAs-s complex (shNS/PAs-s) without specific targets was used as a control. The amount of the control complex injected to the experimental animals was also about 1 mg/kg. shNS refers to GIPZ Non-silencing Lentiviral shRNA Control (catalog No. RHS4346, Dharmacon Co.).

### <Evaluation of Colitis Score>

The evaluation of colitis score was performed by a skilled expert through a blind test. Body-weight reduction, bloody stool, and stool status such as stiffness of stool were observed every two days, and observation was scored. Colitis scale was divided into three parts, body-weight reduction, stool status, and bloody stool and scores were given from 0 points (minimum) to 4 points (maximum). The average score was obtained by averaging the scores for the three parts. Detailed scores for each item are shown in Table 1 below.

TABLE 1

| | Body-weight reduction | Stool status | Bloody stool |
|---|---|---|---|
| 0 | No body-weight | Well-formed granular | No bleeding |
| | reduction | form | |
| 1 | 1 to 5% reduction | | |
| 2 | 5 to 10% reduction | Paste form (not attached | Partial bleeding |
| | | to the anus) or semi- | |
| | | formed granular form | |
| 3 | 10 to 20% reduction | | |
| 4 | 20% or more reduction | Liquid form (attached to | Overall bleeding |
| | | the anus) | |

<Evaluation of Histological Score>

A portion of the intestine of experimental animals (from the cecum to the anus) was removed, and specimens were prepared for histological analysis. Colon samples were fixed in a buffer containing 4% formalin and observed using an H&E staining method. At this time, observation was performed through a blind test. In H&E staining, H refers to hematoxylin, and E refers to eosin. Hemalum is a complex of hematein, an oxidized form of hematoxylin, and aluminum ions, and hemalum binds to DNA and stains the nucleus. Eosin stains microfibers, extracellular fibers, and the like. Experienced pathologists examined the degree of infiltration of inflammatory cells into the intestines and the degree of tissue damage and destruction through a blind test, and the results were scored. Detailed scores for each item are shown in Table 2 below.

TABLE 2

| | Degree of infiltration of | |
|---|---|---|
| | inflammatory cells | Degree of tissue destruction |
| 0 | A small number of immune cells | No damage to mucosal tissue was |
| | was observed in the lamina propria. | observed. |
| 1 | A large number of immune cells | Lymphoepithelial lesions were observed. |
| | was observed in the lamina propria. | |
| 2 | Infiltration of immune cells was | Mucosal erosion or ulcers (in areas where |
| | observed in the submucosal tissue. | pathogens gathered and tissues collapsed) |
| | | were observed. |
| 3 | Infiltration of immune cells was | A wide range of damage was observed in |
| | observed in the transmural layer. | the mucosal tissues, and damage was |
| | | observed deep within the intestinal wall. |

### <Myeloperoxidase (MPO) Assay>

The activity of MPO enzyme was measured, and the degree of infiltration of neutrophil was evaluated based on the MPO activity. Colon samples were homogenized and added to a cold potassium phosphate buffer (50 mM K₂HPO₄, 50 mM KH₂PO₄, pH 6.0) containing 0.5% hexadecyltrimethylammonium bromide. Then, the homogenized samples were subjected to sonication, followed by centrifugation at 17,500 relative centrifugal force (rcf) for 15 minutes. 20 μl of the supernatant of the sample or an MPO standard was mixed with 1 mg/ml of o-dianisidine hydrochloride containing 0.0005% H₂O₂, and absorbance was measured at 450 nm.

### <Measurement of Intracellular Reactive Oxygen Species>

Intracellular reactive oxygen species were measured using a bis-N-methylacridinium nitrate (lucigenin)-ECL method.

Colon lysates were added to a 50 mM phosphate buffer (pH 7.0) containing 1 mM EGTA, 150 mM sucrose, and a protease inhibitor mixture and incubated at 37° C. for 30 minutes. Then, a Krebs-HEPES buffer containing lucigenin (5 μM) and NADPH (100 μM) was added to the lysates to induce reaction. After reaction, the degree of peroxide formation was measured using a luminometer (Lumet LB9507; Berthold Technologies).

### <Measurement of In Vivo Colonic Cytokines>

Colon samples were added to 200 μl of Tissue Protein Extraction Reagent and subjected to vortexing for 1 minute. Subsequently, liquid nitrogen was added to the samples to rapidly freeze the samples. Then, centrifugation was performed at 4° C. and 10,000 g for 15 minutes. The levels of IL-1β, TNF-α, and IL-6 were measured using ELISA.

### <In Vivo Western Blot Analysis>

Colon samples were added to a RIPA buffer containing 10 mM Tris-HCl (pH 8.0), 1 mM EDTA, 140 mM NaCl, 0.1% SDS, 0.1% sodium deoxycholate, 1% Triton X-100, and a protease inhibitor cocktail and incubated at 4° C. for 15 minutes to lyse the samples. After lysis, centrifugation was performed at 4° C. and 14,000 g for 15 minutes to obtain supernatants. The supernatants were subjected to western blot analysis. Proteins present in the supernatants were separated by SDS-PAGE electrophoresis and transferred to a polyvinylidene difluoride (PVDF) membrane. The proteins on the membrane were detected according to the western blot procedure.

### <Induction of Rheumatoid Arthritis Animal Model and Injection of Therapeutic Gene Complex>

DBA/1 female mice were used to prepare an animal model of rheumatoid arthritis. First, bovine type II collagen was mixed with Complete Freund's Adjuvant (CFA) at a concentration of 1 mg/ml, and 100 μl of the solution was injected into the tail of the mouse. Three weeks later, 100 μl of a solution, in which bovine type II collagen and Incomplete Freund's Adjuvant (IFA) were mixed at a concentration of 1 mg/ml, was injected into the tail of the mouse. One week after injection, 50 μg of LPS per mouse was injected intraperitoneally to worsen inflammation. One week after injection of LPS, a gene/carrier complex (shTACE/PAs-s peptoplex) was intravenously injected. After rheumatoid arthritis modeling was completed, the gene/carrier complex (shTACE/PAs-s peptoplex) composed of 20 μg of the gene and 40 μg of the gene carrier (i.e., in an optimal weight ratio of 1:2) was intravenously injected for 5 consecutive days. The amount of the complex treated in the experimental animals was about 1 mg/kg.

### <Images of Rheumatoid Arthritis Animal Model>

One week after the start of rheumatoid arthritis animal modeling, a gene/carrier complex (shTACE/PAs-s peptoplex) was administered to mice and animal experiments were completed after 3 days. The rheumatoid arthritis model mice were anesthetized using carbon dioxide. Images of the forepaw and hind paw of anesthetized experimental animals were obtained.

### <Evaluation of Histological Score for Rheumatoid Arthritis>

Parts of the hind paw and the thigh of experimental animal were removed, and specimens were prepared for histological analysis. The tissue specimens were fixed in a buffer containing 4% formalin and stained using an H&E staining method. Observation of the stained specimens was performed by a skilled pathologist through a blind test procedure. In H&E staining, H refers to hematoxylin, and E refers to eosin. Hemalum is a complex of hematein, an oxidized form of hematoxylin, and aluminum ions, and hemalum binds to DNA and stains the nucleus. Eosin stains microfibers, extracellular fibers, and the like. The degree of progression of inflammatory state and the degree of damage of the cartilage tissue were analyzed by a pathologist's blind test, and the results were scored. Detailed scores for each item are shown in Table 3 below.

[symbol]

3

| | Inflammation | Cartilage damage |
|---|---|---|
| 0 | No inflammation | No destruction |
| 1 | slight thickening of the lining layer | minimal erosion |
| | plus some infiltrating cells in the underlying | limited to single spots |
| | layer | |
| 2 | slight thickening of the lining layer | slight to moderate |
| | plus some infiltrating cells in the underlying | erosion in a limited |
| | layer | area |
| 3 | thickening of the lining layer, an influx | more extensive erosion |
| | of cells in the underlying layer | |
| | and the presence of cells in the synovial | |
| | space | |
| 4 | synovium highly infiltrated with many | general destruction |
| | inflammatory cells | |

### Experiment Results

1. Determination of Optimal Ratio of shTACE/PAs-s Gene/Carrier Complex

To prepare a complex (peptoplex) having excellent prophylactic and therapeutic effects, a gene and a carrier forming the complex must be combined in an optimal ratio. There are various kinds of ratios such as weight ratio, charge ratio, and nitrogen/phosphorous (N/P) ratio. In the present invention, a weight ratio is used. In a weight ratio of X:Y, X may represent the amount (X μg) of the gene and Y may represent the amount (Y μg) of the carrier.

Based on data obtained from agarose gel retardation analysis, the total surface charge of a complex (zeta potential), the average size of a complex, and polydispersity index (PDI) values indicating molecular weight distribution, the optimal ratio of shTACE to PAs-s forming the complex was determined.

1 μg of TACE short hairpin RNA (TACE shRNA or shTACE, SEQ ID NO: 1) was mixed with various amounts of a PAs-s carrier (0.1, 0.5, 1, 2, 3, 4 μg) and incubated at room temperature for 30 minutes to form a gene/carrier complex (shTACE/PAs-s peptoplex), and the total volume was adjusted to 10 μl with deionized/distilled water. Then, the complexes were loaded onto a 0.8% (wt/vol) agarose gel immersed in a 0.5×TBE buffer solution and subjected to electrophoresis at 100 V for 20 minutes.

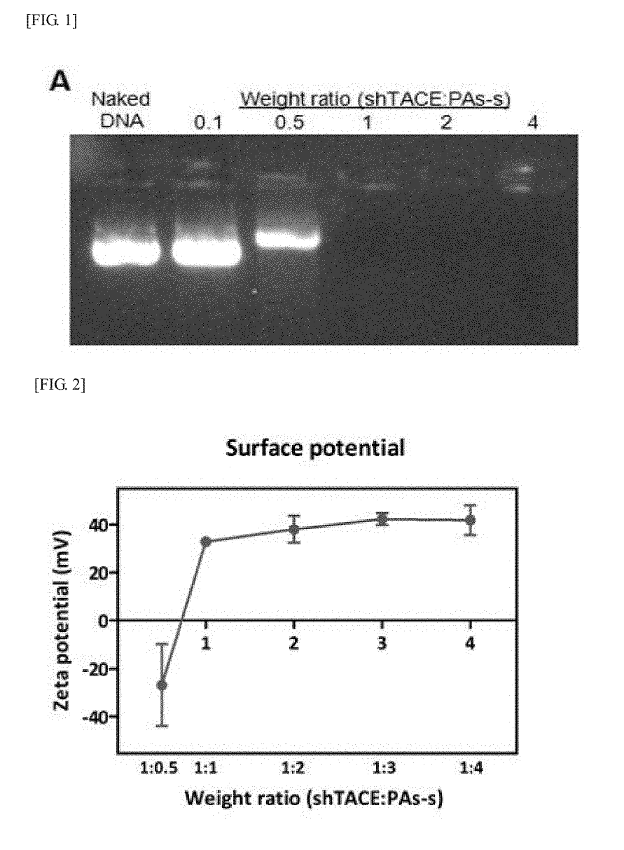

As shown in FIG. 1, when electrophoresis is performed, a negative charge is applied at the upper part of an agarose gel. At this time, only the gene having a negative charge moves to the lower part of the gel due to repulsive force. On the other hand, a complex in which the gene and the carrier are mixed does not move downward but remains in the upper part of the gel. Also, when a weight ratio between the gene and the carrier is 1:0.1 or 1:0.5, a complex is not well formed and the gene is moved downward. In this case, the overall polarity of the complex is negatively charged because the amount of the carrier is less than the amount of the gene, resulting in the complex moving on the gel. On the other hand, when a weight ratio was 1:1 or more, the complex was well formed, and the complexes having a weight ratio of 1:1 or more were further tested as candidates for the optimal weight ratio.

5 μg of shTACE was mixed with various amounts of a PAs-s carrier to form gene/carrier complexes (shTACE/PAs-s peptoplex) having various weight ratios (shTACE:PAs-s=1:0.5, 1:1, 1:2, 1:3, and 1:4), and the total volume of the complex was adjusted to 800 μl with a buffer solution. The surface charge (zeta potential), size, and PDI value of the gene/carrier complexes (peptoplexes) were measured using Zetasizer Nano ZS (Malvern).

As shown in FIG. 2, the surface charge of the complex formed at a weight ratio of 1:0.5 (shTACE:PAs-s) showed a negative charge. This indicated that at a weight ratio of 1:0.5, a complex was not formed properly, and as a result, the surface charge became negative due to the negative charge of the gene. On the other hand, when the weight ratio was more than 1:1, the surface charge exhibited a positive charge. As the amount of the positively charged gene carrier increased, the surface charge increased to about 40 mV. Therefore, according to the results of the surface charge measurement, complexes having a weight ratio of gene to gene carrier of 1:1 or more were selected as candidates of the complex having the optimal weight ratio. These results were similar to those obtained from electrophoresis experiments.

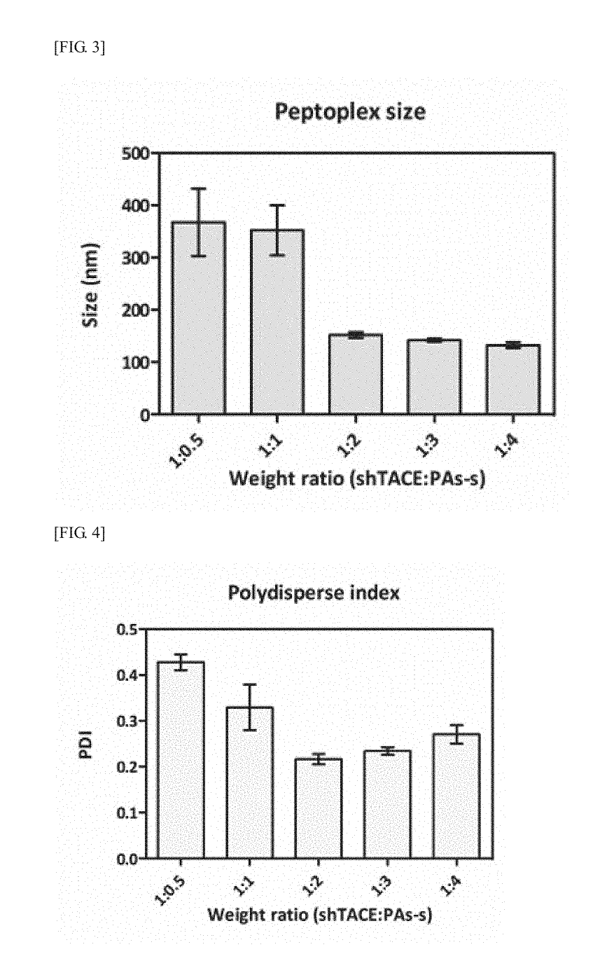

However, the average size and PDI value of the gene/carrier complex having a weight ratio of 1:1 were too large when compared to the gene/carrier complexes having a weight ratio of 1:2 or more. Thus, the complex having a weight ratio of 1:1 was determined to be inappropriate [FIG. 3]. Preferably, the size of the complex is about 200 nm and a PDI value is about 0.2. Since the gene/carrier complex having a weight ratio of 1:2 or more exhibited the above-mentioned size and PDI value, a ratio of 1:2 or more was determined as an optimal ratio. Although the difference was small, at a weight ratio of 1:2, the PDI value was closer to 0.2 than at a weight ratio of 1:3 or 1:4 [FIG. 4]. Although the weight ratios of 1:2, 1:3, and 1:4 were all appropriate, the optimal ratio between the shTACE and the PAs-s carrier was determined to be 1:2 to minimize the total amount of a complex.

In experiments to determine the optimal weight ratio between the gene and the gene carrier (shTACE:PAs-s), the amount of the gene was fixed at 1 μg, various amounts of the carrier were added to prepare the gene/carrier complexes (shTACE/PAs-s peptoplexes) having various weight ratios (shTACE:PAs-s=1:1, 1:2, 1:3, 1:4, 1:6, and 1:8), and properties and effects thereof were examined

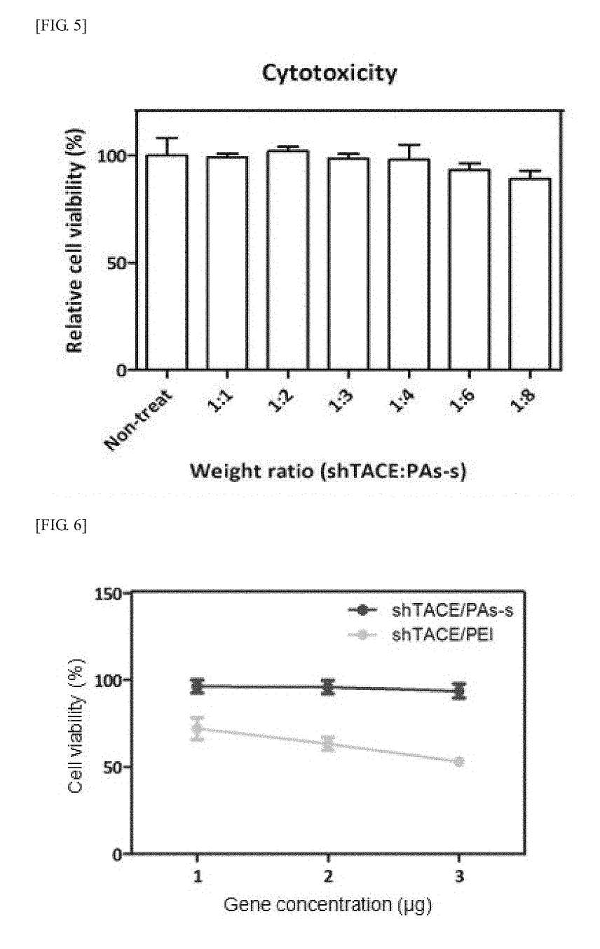

In cytotoxicity tests, no toxicity was observed at all weight ratios [FIG. 5]. However, as the amount of the gene carrier increased, cell viability tended to decrease slightly. Therefore, the total amount of the complex was minimized, and the optimal ratio was selected as 1:2.

To assess the toxicity of the complex (shTACE/PAs-s peptoplex) with the optimal weight ratio of 1:2, mouse-derived macrophages (RAW 264.7 cells) were treated with various amounts of the complex (1, 2, and 3 μg per ml of complete medium (CM)). According to the optimal weight ratio of 1:2, the shTACE/PAs-s complex was prepared. For example, when 1 μg of the shTACE gene was used, 2 μg of the PAs-s carrier was used, when 2 μg of the shTACE gene was used, 4 μg of the PAs-s carrier was used, and when 3 μg of the shTACE gene was used, 6 μg of the PAs-s carrier was used. Then, RAW 264.7 cells were treated with the prepared complexes. In this case, a shTACE/polyethylenimine (shTACE/PEI) complex was used as a control.

Mouse-derived macrophages (RAW 264.7 cells) treated with the shTACE/PAs-s complexes having the optimal weight ratio of 1:2 exhibited little cytotoxicity. When compared to the control complex (shTACE/PEI), higher cell activity was observed in cells treated with the shTACE/PAs-s [FIG. 6]. In addition, shTACE was used in amounts of 1 μg, 2 μg, and 3 μg to form complexes (That is, 1 μg of shTACE and 2 μg of PAs-s, 2 μg of shTACE and 4 μg of PAs-s, and 3 μg of shTACE and 6 μg of PAs-s were used, respectively, to form complexes). Then, cells were treated with the shTACE/PAs-s complexes. In this case, cytotoxicity was scarcely observed.

2. Comparison of Effects of Existing shTACE/rPOA Complex and Novel shTACE/PAs-s Complex

In an existing complex (old peptoplex, existing shTACE/rPOA, Non-Patent Document 1), the shTACE plasmid having an existing sequence described in Non-Patent Document 1 is difficult to amplify using Maxiprep, and thus the amplification yield of the shTACE plasmid is very low. The carrier used in the existing complex is reducible poly(oligo-arginine). The existing rPOA carrier is a polymeric nonviral carrier formed by polymerization reaction of a monomer of Cys-(9×Arg)-Cys, but the basic salt form of the monomer is TFA salt. Since TFA salt may cause unexpected side effects in the body, the acetate of disulfide-linked poly(oligo-arginine) (PAs-s) in which the salt of the monomer is substituted with acetate is used as a novel complex. In the present invention, a novel complex capable of overcoming the disadvantages of the existing complex was prepared. According to the present invention, a gene sequence (i.e., the sequence of shTACE plasmid) and the salt form of a monomer used as a carrier were improved. The advantages of using the novel complex (novel peptoplex, shTACE/PAs-s) are as follows.

To compare the toxicity of the existing complex and the novel gene/carrier complex, cells were treated with the gene/carrier complexes having an optimal weight ratio of 1:2 (gene:carrier). The gene was used as an amount of 2 μg or 4 μg per ml of complete medium (CM). Based on the optimal weight ratio of 1:2, the existing and novel gene/carrier complexes were prepared. For example, when shTACE was used in an amount of 2 μg, the carrier was used in an amount of 4 μg, or when shTACE was used in an amount of 4 μg, the carrier was used in an amount of 8 μg. Then, cells were treated with the prepared complexes.

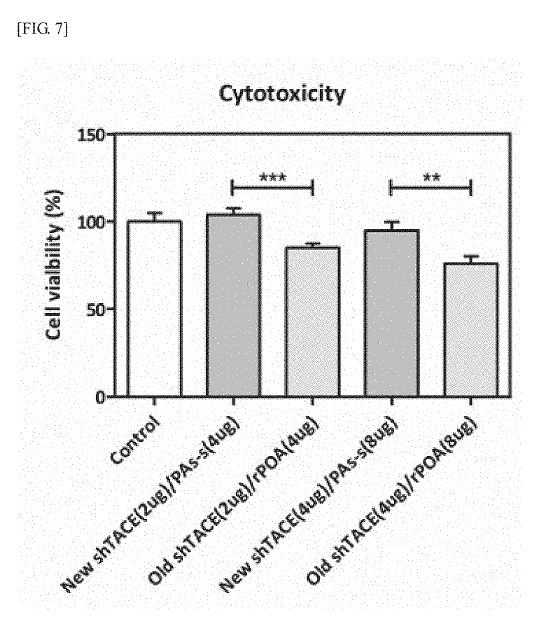

Mouse-derived macrophages (RAW 264.7 cells) were treated with the existing shTACE/rPOA complex (old peptoplex) or the novel shTACE/PAs-s complex (new peptoplex, shTACE/PAs-s) of the present invention. After 24 hours, both complexes were compared in terms of cytotoxicity. As shown in FIG. 7, despite the fact that both the existing and novel complexes contained the same amount (2 μg or 4 μg) of shTACE gene, the novel shTACE/PAs-s complex (new peptoplex) exhibited much less cytotoxicity compared to the existing shTACE/rPOA complex (old peptoplex). Specifically, in both cases of the existing complex and the novel gene/carrier complex, according to the optimal weight ratio 1:2, when shTACE was used in an amount of 2 μg, the carrier was used in an amount of 4 μg, and when shTACE was used in an amount of 4 μg, the carrier was used in an amount of 8 μg. In both cases where 2 μg of shTACE was used and 4 μg of shTACE was used, compared to the existing gene/carrier complex, relative cell viability was significantly higher in the novel gene/carrier complex-treated cells (statistical significance using two-tailed Student's t-test: *P<0.05, **P<0.01, and ***P<0.001). Relative cellular metabolic activity was measured by MTT assay.

Cells were treated with the existing complex or the novel complex of the present invention and incubated for 24 hours. After incubation, the extent to which the expression of TACE mRNA was reduced by the action of short hairpin RNA (shRNA) was measured. Both complexes contain shTACE, which down-regulates TACE expression through an RNA interference system (although shTACE genes contained in the two complexes have different sequences), so that TACE expression is reduced when the complexes enter into cells and are expressed. Mouse-derived macrophages (RAW 264.7 cells) were treated with each complex and incubated for 24 hours. According to the optimal weight ratio, in both cases of the existing and novel shTACE, 1 μg of shTACE gene was mixed with 2 μg of rPOA or PAs-s 2 to form the complex. Then, cells were treated with the formed complex. After 24 hours, RNA was isolated from each group of cells, and complementary DNA (cDNA) was synthesized using the isolated RNA. Then, mRNA level of TACE was measured using RT-PCR. GAPDH, a structurally expressed gene, was used as an endogenous control.

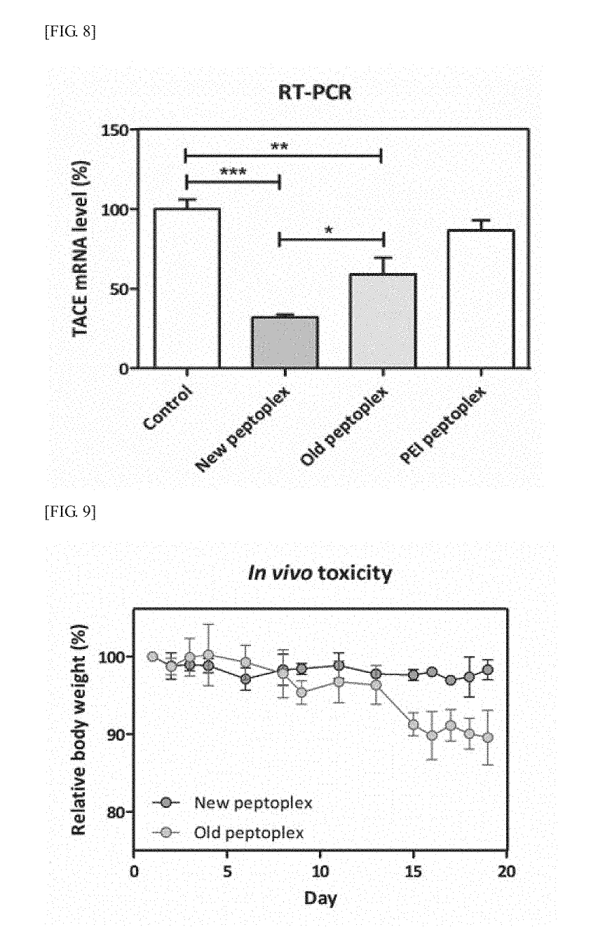

As a result, based on the TACE mRNA level of a control group in which cells were not treated with shTACE, the TACE mRNA level of the novel complex-treated cells was significantly lower than the TACE mRNA level of the existing complex-treated cells [FIG. 8]. These results indicate that the transfection efficiency of the novel shTACE/PAs-s complex (new peptoplex) is higher than that of the shTACE/rPOA complex (old peptoplex). That is, it is judged that the amount of introduced shTACE is larger in the novel complex-treated cells, and thus the expression of TACE mRNA is further decreased due to the increased role of the shTACE.

Basically, gene therapy agents injected into experimental animals should not have toxicity. When a gene therapy agent itself is toxic, it is impossible to inject the agent into the body, and unexpected negative experimental results may be obtained. Thus, cytotoxicity must be minimized. Minor toxicity also affects experimental results, so that the experimental results may be misinterpreted.

The existing and novel complexes were injected into the peritoneal cavity of C57BL/6 mouse, respectively. After a period of time, body weight was measured, and toxicity was checked. When the existing shTACE/rPOA complex (old peptoplex) was injected, body weight dropped dramatically. On the other hand, when the same amount of the shTACE/PAs-s complex (new peptoplex) according to the present invention was injected, body weight was relatively well maintained [FIG. 9]. When the mice were observed for 20 days after the first injection, in the case of mice injected with the existing complex, body weight decreased by 10% in two weeks. It can be concluded that these results are caused by the intrinsic toxicity of the existing complex.

These results suggest that in the case of the existing complex, endogenous endotoxins, which have not been removed during the amplification of the shTACE plasmid containing an existing sequence by a Miniprep method, and the trifluoroacetic acid (TFA) salt form of the rPOA carrier are toxic to experimental animals. To amplify shTACE containing an existing sequence, which is difficult to amplify using Maxiprep, it is inevitable to use Miniprep. Since the existing sequence is changed in the novel shTACE, the novel shTACE may be amplified with high yield by Maxiprep. In addition, since endotoxins are automatically removed during the Maxiprep procedure, the shTACE does not cause abnormal toxic reactions when the novel complex is injected into the body. Furthermore, since the salt form of the PAs-s carrier is a form of acetate, compared to the existing the TFA salt form, it is possible to minimize abnormal reactions that may be induced in vivo.

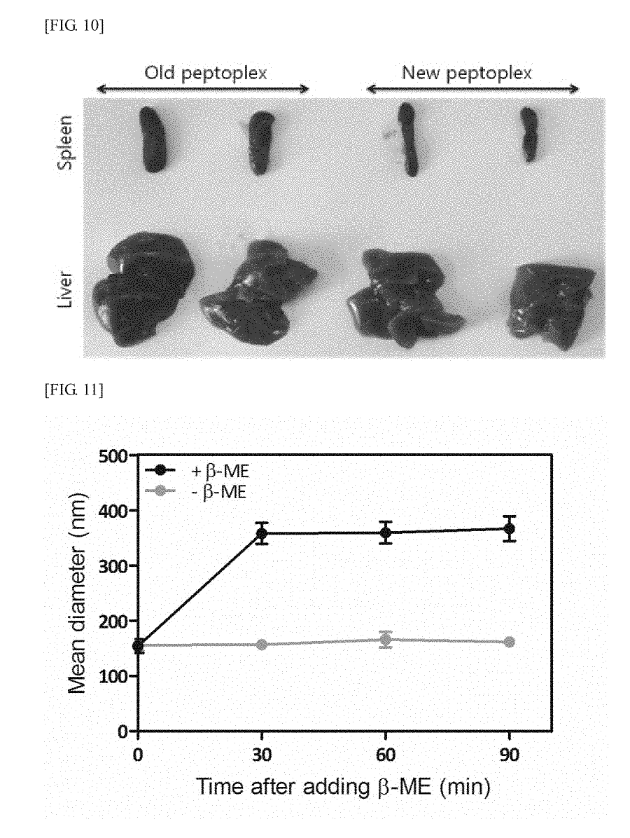

In addition, the size of liver and spleen increases in experimental animals injected with toxic substances. 19 days after the first injection of the existing complex or the novel complex, experimental animals were sacrificed to obtain liver and spleen tissues. When the tissues were examined visually, it was confirmed that the spleens of experimental animals injected with the existing complex (old peptoplex) were enlarged [FIG. 10]. In addition, in the case of the existing complex (old peptoplex), the average weight of liver was 1.5 g. On the other hand, in the case of the novel shTACE/PAs-s complex (new peptoplex), the average weight of liver was 1.35 g. Compared to the novel complex-injected animals, the existing complex-injected animals weighed less. Thus, considering liver weight relative to body weight, the difference between the two groups may be more noticeable. These results indicate that the existing complex is toxic.

Therefore, the novel complex showed better results than the existing complex in terms of cytotoxicity, animal toxicity, transfection efficiency (or efficiency of silencing TACE expression), and the like.

3. Evaluation of Anti-Inflammatory Effect of shTACE/PAs-s Complex (Cell Experiments)

The PAs-s carrier is a polymer of poly(arginine) in which a monomer of nine D-arginines including cysteine groups at both ends thereof is a repeating unit and the monomers are linked via disulfide bonds between cysteines. The disulfide bond is cleaved by a reducing agent and reduced to a thiol group (—SH). Examples of a reducing agent include β-mercaptoethanol and the like. When not treated with β-mercaptoethanol (−β-ME), the size of the shTACE/PAs-s complex remains constant until 2 hours after formation of the complex. On the other hand, when treated with β-mercaptoethanol (+β-ME), disulfide bonds in the carrier are reduced and loosened by the reducing agent. As a result, the interaction between the gene and the carrier is loosened, and the size of the gene/carrier complex is increased [FIG. 11]. This is an experiment that mimics an in vivo process which may be mediated by glutathione in the cytoplasm. Glutathione is mainly present in cells, especially in the cytoplasm, rather than in extracellular regions. The difference in glutathione concentration inside and outside of the cell gives rise to a high redox potential. That is, glutathione present in high concentration in cytoplasm in vivo has ability to reduce. Accordingly, due to the reducing action of the glutathione, the disulfide bonds of the carrier included in the shTACE/PAs-s complex are loosened and become disrupted in the cell. As a result, the interaction between the carrier and the shTACE gene is weakened, and the shTACE gene is released into the cytoplasm.

Transfection is a process of introducing DNA into cultured animal cells and expressing DNA in cells. The transfection efficiency of a PAs-s carrier was determined by the expression level of luciferase enzyme. Luciferase enzyme is expressed from a luciferase plasmid (pLuci) in cells. The enzymatic activity of luciferase oxidizes luciferin to emit light energy, and thus pLuci acts as a reporter gene that indirectly indicates the expression level of a foreign gene introduced into cells. 1 μg of luciferase plasmid (pLuci) was mixed with 2 μg of the PAs-s carrier and incubated at room temperature for 30 minutes to form a pLuci/PAs-s complex. Then, mouse-derived macrophages (RAW 264.7 cells) were treated with the complex. After 48 hours, the relative reactivity between cells of each group and a luciferase substrate solution supplied from the outside was measured, and the degree of pLuci expressed in cells was measured.

The degree of pLuci expression and the degree of reaction between a luciferase substrate and an enzyme in the cell are proportional. A PEI carrier was used as a control. The transfection efficiency of the PAs-s carrier was higher than that of the PEI carrier.

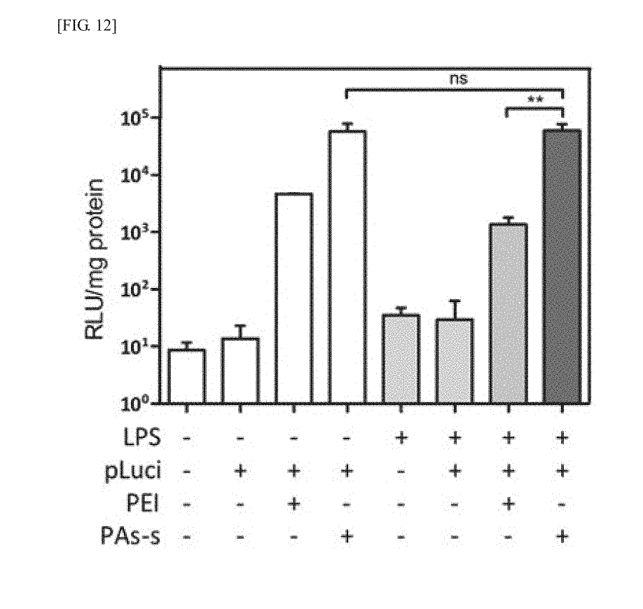

Regardless of whether lipopolysaccharide (LPS) treatment was performed, the transfection efficiency of the PAs-s complex remained high [FIG. 12]. To mimic inflammatory conditions in vitro, macrophages are treated with LPS, which causes macrophages to become activated. The results of luciferase assay show that the transfection efficiency of the PAs-s carrier to macrophages is high in an inflammatory state as well as in a non-inflammatory state. These results indicate that the shTACE/PAs-s complex including the PAs-s carrier may be effectively delivered to macrophages in an inflammatory state.

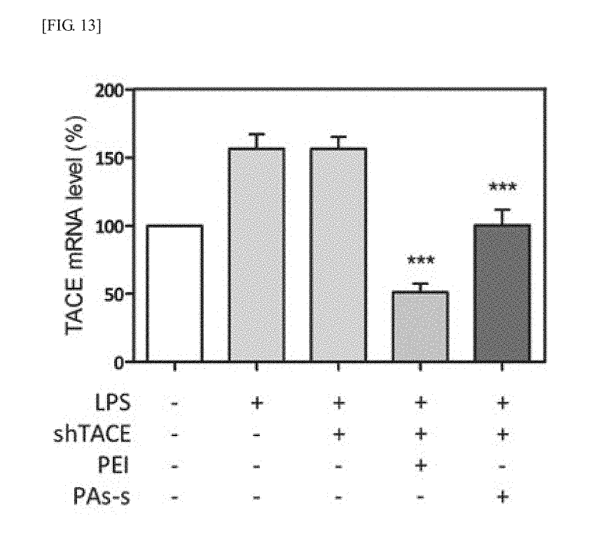

RAW 264.7 cells, a mouse macrophage cell line, were treated with LPS, and then treated with the shTACE/PAs-s complex prepared by mixing the shTACE therapeutic gene and the PAs-s carrier, followed by incubation for 48 hours. Thereafter, when TACE mRNA expression was measured, the level of TACE mRNA was reduced by the role of shTACE inhibiting the expression of TACE mRNA [FIG. 13]. The PAs-s carrier allowed the shTACE gene to enter cells, and the shTACE gene was expressed in the cells, resulting in a decrease in TACE mRNA levels by a TACE interference effect. On the other hand, when only the shTACE gene was introduced without the carrier, only a small amount of the shTACE gene entered the cells due to the repulsive force between the gene and the cell membrane, and the effect of interfering with TACE expression was hardly observed. As a result, it was confirmed that the expression level of TACE mRNA in the shTACE gene-treated group without the carrier was similar to that of TACE mRNA in the LPS-treated group.

TACE mRNA levels were higher in an LPS-treated group than in an LPS-untreated group. This result indicates that TACE mRNA expression increases in an inflammatory state. That is, these results indicate that it is desirable to select TACE as a target gene for treatment of inflammatory diseases overexpressing TACE.

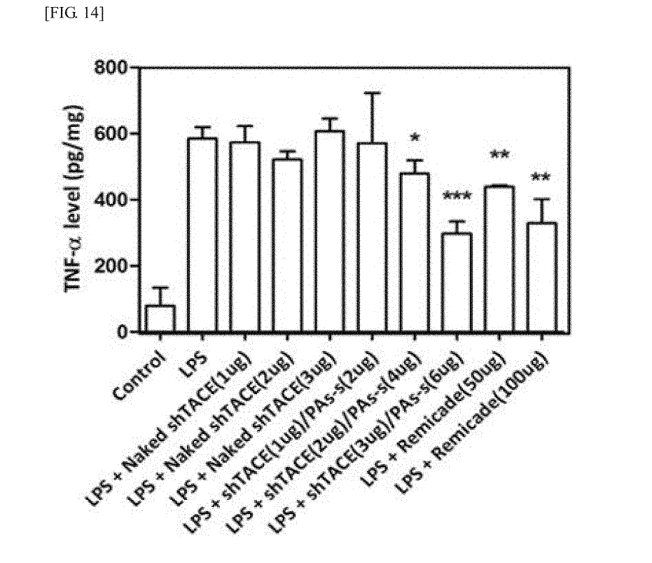

When RAW 264.7 cells, a mouse macrophage cell line, were treated with LPS, macrophages became activated. Compared to a non-treated group, the level of soluble TNF-α released from macrophages was higher in an inflammatory state [FIG. 14]. These results indicate that TACE is overexpressed in an inflammatory state and that inflammation is exacerbated by the increased water-soluble TNF-α(in vitro). After LPS treatment, when cells were treated with a complex including the shTACE therapeutic gene and a gene carrier and incubated for 48 hours, TACE mRNA expression was down-regulated and the amount of soluble TNF-α was reduced. Due to the role of the carrier, the shTACE gene may effectively enter the cell, and the shTACE gene introduced into the cell is expressed, interfering with TACE mRNA expression. On the other hand, after the LPS treatment, when cells were treated with shTACE alone without the gene carrier, a small amount of shTACE was introduced into the cells, and as a result, the effect of interfering with TACE expression was hardly observed. In addition, there was no significant difference in the amount of soluble TNF-α compared with the group treated with LPS alone. Similar results were obtained when increasing the amount of shTACE treated.

The decrease in the amount of soluble TNF-α cytokine was proportional to the amount of shTACE/PAs-s complex. That is, these results demonstrate that the shTACE/PAs-s complex effectively reduces the level of TNF-α in cells to treat inflammatory diseases. In addition, after LPS treatment, when cells were treated with 50 μg and 100 μg of Infliximab (Remicade), a monoclonal antibody targeting TNF-α, the amount of soluble TNF-α was decreased in proportion to the concentration. After LPS treatment, the inhibitory effects of the shTACE/PAs-s complex and Infliximab (Remicade) on the level of TNF-α were compared. Cells were treated with a complex including 3 μg of shTACE or 100 μg of Infliximab (Remicade), and the amount of soluble TNF-α was measured in each case. The amount of soluble TNF-α was similar in both cases. These results suggest that the shTACE gene may produce a similar effect in a smaller amount than Infliximab (Remicade), showing that the complex including the shTACE gene is superior in terms of drug dose.

4. Measurement of TACE Expression after Injection of shTACE/PAs-s Complex in Animal Model for Ulcerative Colitis

The gene/carrier complex (shTACE/PAs-s peptoplex) having an optimal weight ratio of 1:2 was intravenously injected for two consecutive days. After 6 and 11 days, liver and spleen of experimental animals were obtained and the expression level of TACE protein was measured. Upon intravenous injection, according to the optimal weight ratio of 1:2, 20 μg of the shTACE gene and 40 μg of the PAs-s carrier were mixed in PBS and incubated for 30 minutes to form a complex. When liver and spleen obtained on the 6th day were examined, TACE expression was reduced in experimental animals injected with a shTACE (20 μg)/PAs-s (40 μg) complex. When liver and spleen obtained on the 11th day were examined, TACE expression was reduced in experimental animals injected with a shTACE (10 μg)/PAs-s (20 μg) complex or a shTACE (20 μg)/PAs-s (40 μg) complex [FIG. 15]. These results indicate that the intravenously injected shTACE/PAs-s complex works well after circulation in the body and reduces TACE expression.

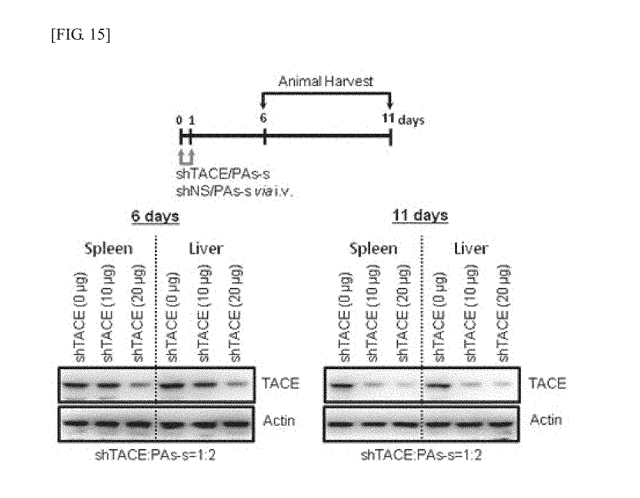

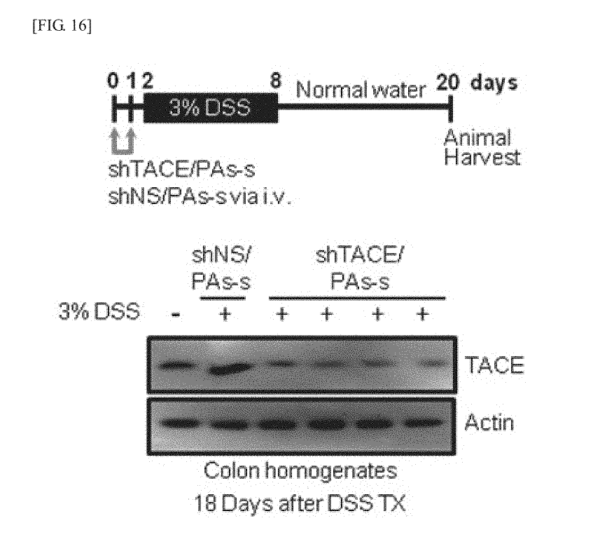

To produce an animal model for ulcerative colitis, the gene/carrier complex (shTACE/PAs-s peptoplex) was first intravenously injected into experimental animals for two consecutive days, and 3% dextran sodium sulfate (DSS) was supplied to the experimental animals. The protein expression of TACE increased in the ulcerative colitis model animals supplied with 3% DSS. These results confirm the increased expression of TACE in inflammatory conditions of ulcerative colitis in vivo. To determine whether the shTACE/PAs-s complex could prevent ulcerative colitis, the shTACE (20 μg)/PAs-s (40 μg) complex was injected to experimental animals prior to performing ulcerative colitis modeling, and then ulcerative colitis modeling was performed. As a result, TACE expression in the colon, the main target of this experiment, was reduced [FIG. 16].

5. Evaluation of Preventive Effect of shTACE/PAs-s Complex in Ulcerative Colitis Model

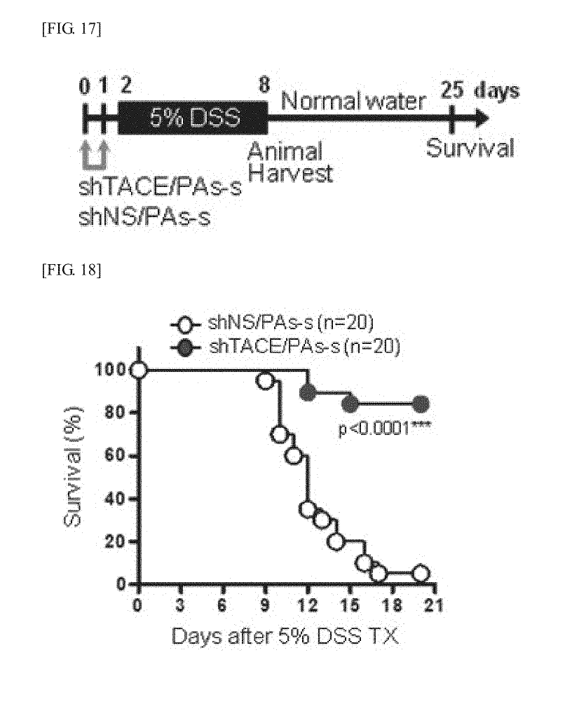

To determine whether the shTACE/PAs-s complex could prevent ulcerative colitis, shTACE and the gene carrier (PAs-s) were mixed and incubated at room temperature for 30 minutes to form the complex (shTACE/PAs-s). The complex was administered to experimental animals over two days via intravenous infusion. Then, to perform acute ulcerative colitis modeling, water containing 5% dextran sodium sulfate (DSS) was supplied to the animals [FIG. 17]. Upon intravenous injection, according to the optimal weight ratio, 20 μg of shTACE and 40 μg of PAs-s (shTACE(20 μg)/PAs-s(40 μg)) were mixed in PBS and incubated for 30 minutes to form a complex. This corresponds to treating the complex in an amount of 1 mg/kg per animal in a single intravenous injection

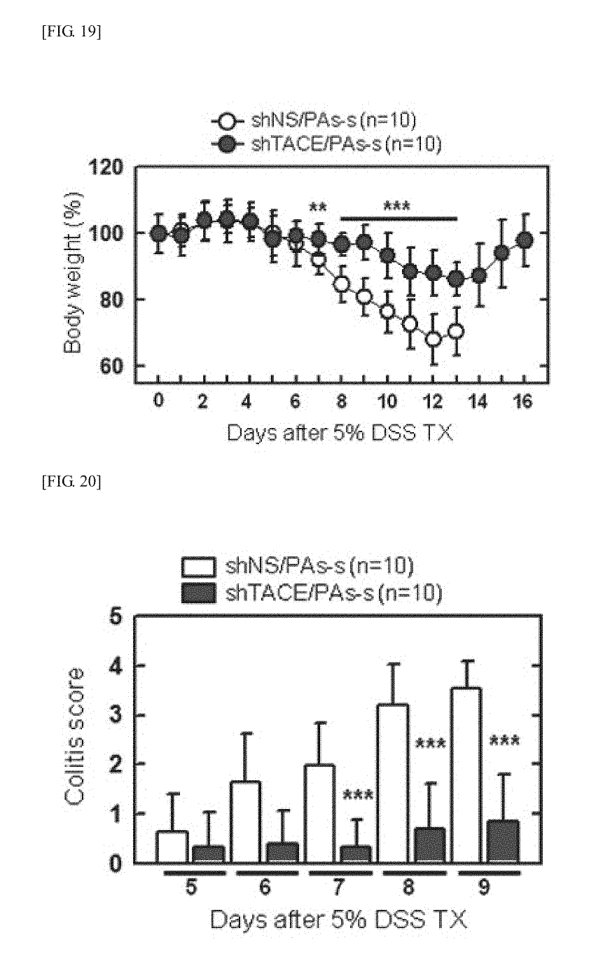

In terms of prevention, when compared with animals injected with nonspecific shRNA (shNS)/PAs-s, animals injected with the shTACE/PAs-s complex showed significantly higher survival rates and body weights. In various inflammatory conditions, including ulcerative colitis, the body weight of experimental animals is drastically reduced, thereby reducing the survival rate. The survival rate of the shTACE/PAs-s complex-injected experimental animals was higher than that of the control group, and the body weight remained constant [FIGS. 18 and 19]. Colitis scale was divided into three parts, body-weight reduction, stool status such as stiffness of stool, and bloody stool, and the average score was obtained by averaging the scores for the three parts. TACE expression was decreased by the action of the shTACE gene in experimental animals in which the shTACE/PAs-s complex was pre-injected for prevention purposes. As a result, the stool of the experimental animals was in good condition, bloody stool was reduced, and body weight was maintained normally. Compared to a nonspecific shRNA (shNS)/PAs-s complex-injected group, the colitis score was significantly lower in the shTACE/PAs-s complex-injected animals [FIG. 20].

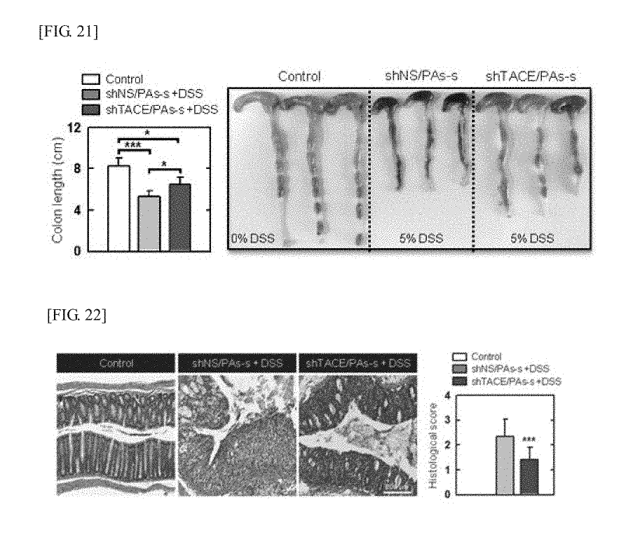

After completion of the animal modeling, experimental animals were sacrificed, and colon was obtained. Then, the length of the colon was measured, and histological analysis was performed on the colon. In an acute ulcerative colitis group treated with a non-specific shRNA (shNS)/PAs-s complex, inflammation progressed, colon contracted, and bleeding reddened the colon. In the case of a group treated with the shTACE/PAs-s complex for prevention purposes, compared to the non-specific shRNA (shNS)/PAs-s complex-treated group, a relatively long colon was observed. In addition, the colon color of the shTACE/PAs-s complex-treated group was similar to that of a normal control group. From these results, it was confirmed that mice were prevented from acute ulcerative colitis due to the preventive effect of the complex [FIG. 21]. When histological analysis was performed on the colon tissue, compared to the non-specific shRNA (shNS)/PAs-s complex-treated group, the shTACE/PAs-s complex-treated group exhibited a less collapsed colon structure, and phenotypes indicating the degree of inflammation such as enterocyte loss, crypt inflammation, the degree of lamina propria mononuclear cells, and epithelial hyperplasia were weak in the shTACE/PAs-s complex-treated group. That is, in the shTACE/PAs-s complex-treated group, histological scores indicating the degree of inflammation were low [FIG. 22].

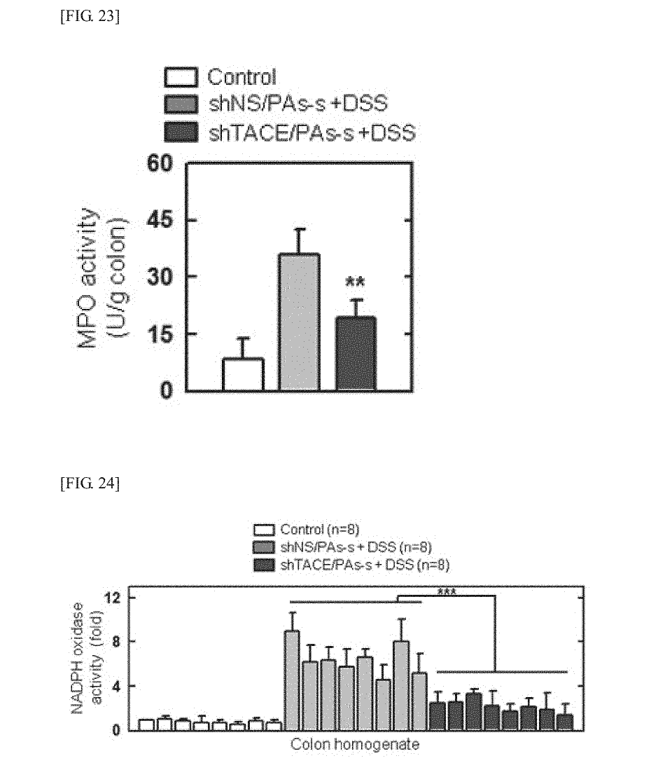

The activity of myeloperoxidase (MPO) enzyme was measured, and based thereon, the degree of neutrophil infiltration into the colon tissue was measured. Inflammation increases the penetration of macrophages, neutrophils, and other immune cells into the colon tissue. The extent of inflammation may be indirectly determined by the degree of penetration. In an acute ulcerative colitis model, the penetration of neutrophils, one type of immune cells, was increased. When compared to a shNS-treated group, in the shTACE/PAs-s complex-treated group, immune cell penetration decreased [FIG. 23].

The amount of intracellular reactive oxygen species was measured in ulcerative colitis experimental animals using a lucigenin (bis-N-methylacridinium nitrate)-ECL method. Based on the activity of NADPH oxidase, the amount of intracellular reactive oxygen species was determined. As a result, in the shTACE/PAs-s complex-treated group, the amount of reactive oxygen species was reduced compared to the nonspecific shRNA (shNS)-treated group [FIG. 24]. 50 mM phosphate buffer (pH 7.0), 1 mM EGTA, 150 mM sucrose, and a protease inhibitor mixture were mixed with a Krebs-HEPES buffer solution containing 5 μM lucigenin to prepare electron acceptors. NADPH (100 μM) acts as an electron donor. The amount of reactive oxygen species was determined by the amount of superoxide generated by the reaction.

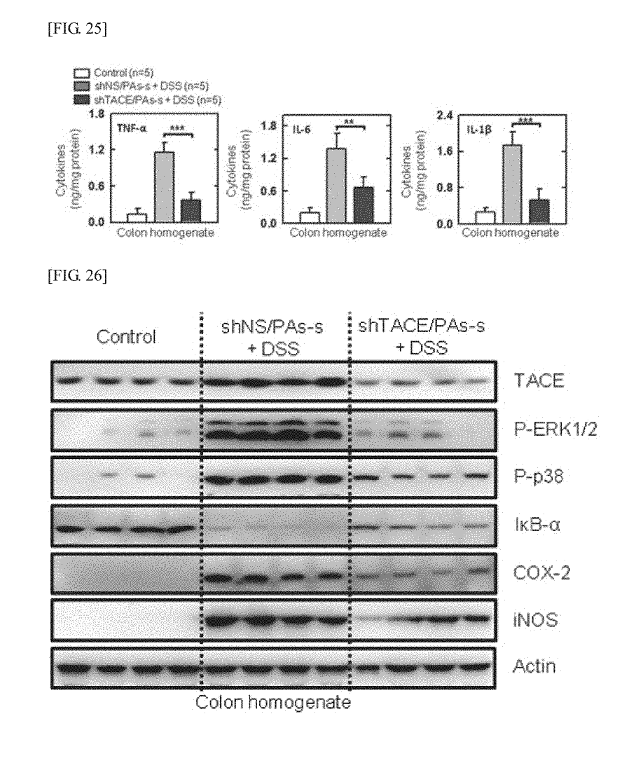

In colon samples, the concentrations of inflammatory cytokines including IL-1β, TNF-α, and IL-6 were measured using ELISA. The main role of TACE is to promote the secretion of soluble TNF-α through transmembrane TNF-α. Therefore, it was confirmed that the amount of TNF-α decreased due to the decrease of TACE expression by the shTACE/PAs-s complex. In addition, it was confirmed that the amount of other types of inflammatory cytokines also decreased [FIG. 25].

Both acute and chronic ulcerative colitis are regulated by mitogen-activated protein kinases (MAPK)-NF-κB inflammatory signaling. In the nonspecific shRNA (shNS)-treated ulcerative colitis model, the expression of various kinds of proteins related to the intestinal inflammatory pathways was increased compared to the normal experimental animals. On the other hand, in the shTACE/PAs-s complex-treated ulcerative colitis model, there was little change in the expression of inflammation-related proteins as in normal animal models [FIG. 26]. In addition, the expression levels of ERK1/2, p38 MAPK, COX-2, and iNOS were down-regulated compared to the nonspecific shRNA (shNS)-treated group. High IκB-α expression was observed due to degraded NF-κB. In an inflammatory state, inhibitory κBα (IκB-α) is associated with NF-κB. Conversely, in a non-inflammatory state, phosphorylation of IkB-α is induced by IkB-α kinase (IKK) and IκB-α is separated from NF-κB, so the amount of IκB-α increases in the non-inflammatory state.

When shTACE was injected with the carrier prior to modeling ulcerative colitis with DSS, the expression of inflammation related proteins was reduced. In addition, the basal level of TACE was increased in the ulcerative colitis model compared with the normal animal model, and the expression level of TACE was decreased in the shTACE/PAs-s complex-treated group. Thus, the amount of TACE protein expression was consistent with the TACE mRNA level described above.

6. Evaluation of Therapeutic Effect of shTACE/PAs-s Complex in Ulcerative Colitis Model

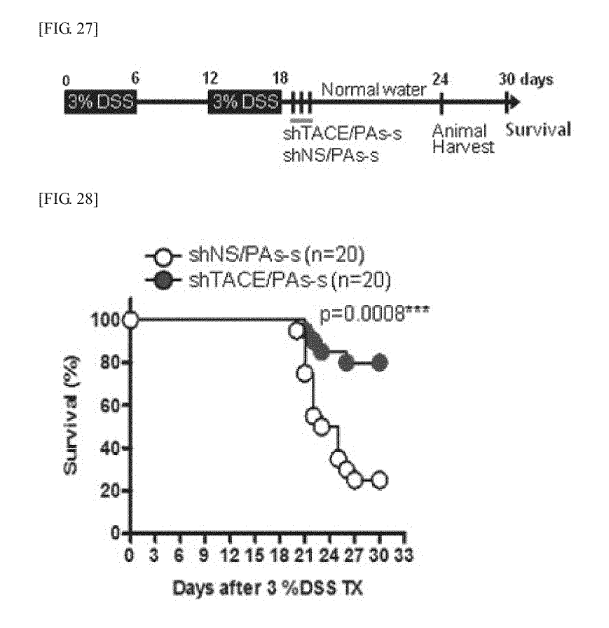

To perform chronic ulcerative colitis modeling, water containing 3% dextran sodium sulfate (DSS) was supplied to experimental animals every other week for 3 weeks. After the ulcerative colitis modeling was completed, the shTACE/PAs-s complex was intravenously injected. At this time, the intravenous infusion was carried out for three consecutive days in the same amount (shTACE (20 μg)/PAs-s (40 μg)) [FIG. 27]. For an intravenously injected complex, according to the optimal weight ratio of 1:2, 20 μg of the shTACE gene and 40 μg of the PAs-s carrier were mixed in PBS and incubated for 30 minutes to form a complex. This corresponds to treating the complex in an amount of 1 mg/kg per animal in a single intravenous injection.

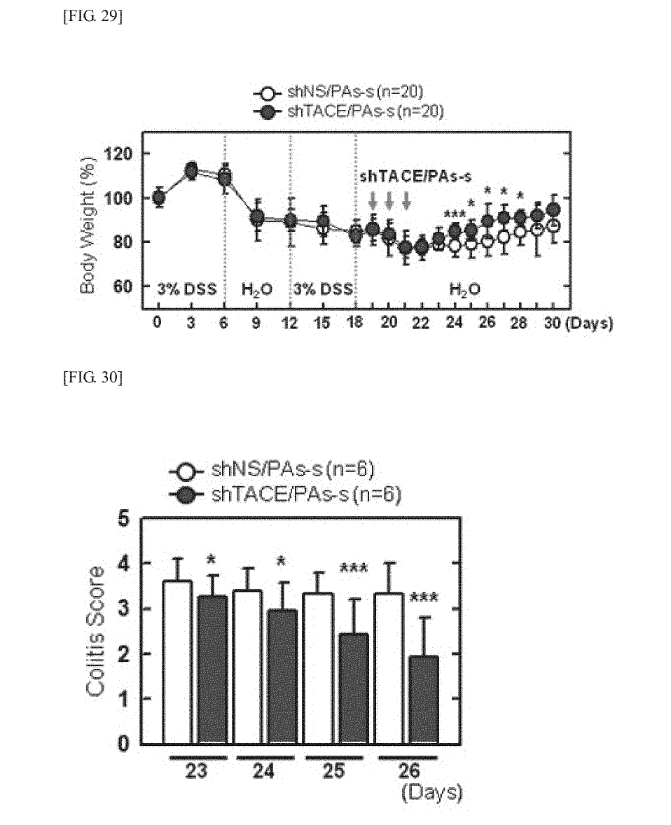

After completion of chronic ulcerative colitis modeling, the shTACE/PAs-s complex was injected to the animal model. In the group injected with the shTACE/PAs-s complex, a survival rate was higher, body weight reduction was smaller, and a colitis score value, which indicated inflammation degree and was evaluated by phenotypes such as bleeding at the rectum, stool status, and the like, was low when compared to a nonspecific shRNA (shNS)-injected group [FIGS. 28 to 30].

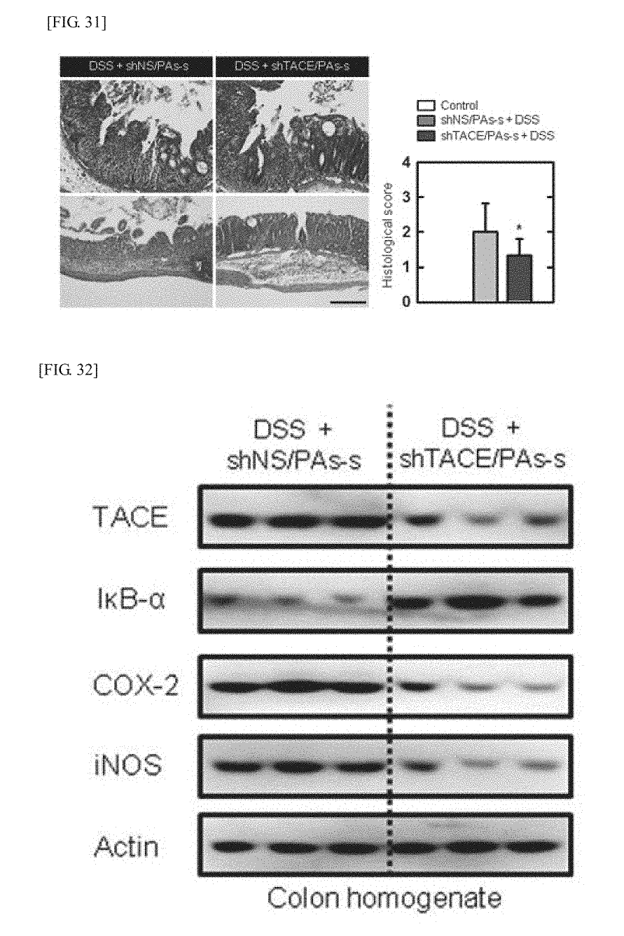

Histological analysis confirmed that the inflammatory state was alleviated by suppression of TACE expression. Immune cell penetration and tissue destruction were observed, and scores were significantly decreased [FIG. 31]. In addition, western blot analysis confirmed that TACE expression, which was overexpressed in the colon, was reduced by shTACE/PAs-s treatment in the animal model. It was also confirmed that expression of inflammation-related proteins (COX-2 and iNOS) was also decreased. On the other hand, the protein level of IκB-α was increased [FIG. 32]. In the non-inflammatory state, phosphorylation of IκB-α is induced by IkB-α kinase (IKK) and IκB-α is separated from NF-κB, so the amount of IκB-α increases. This was confirmed by Western blotting. [FIG. 32].

7. In Vivo Distribution of Intravenously Injected shTACE/PAs-s Complex

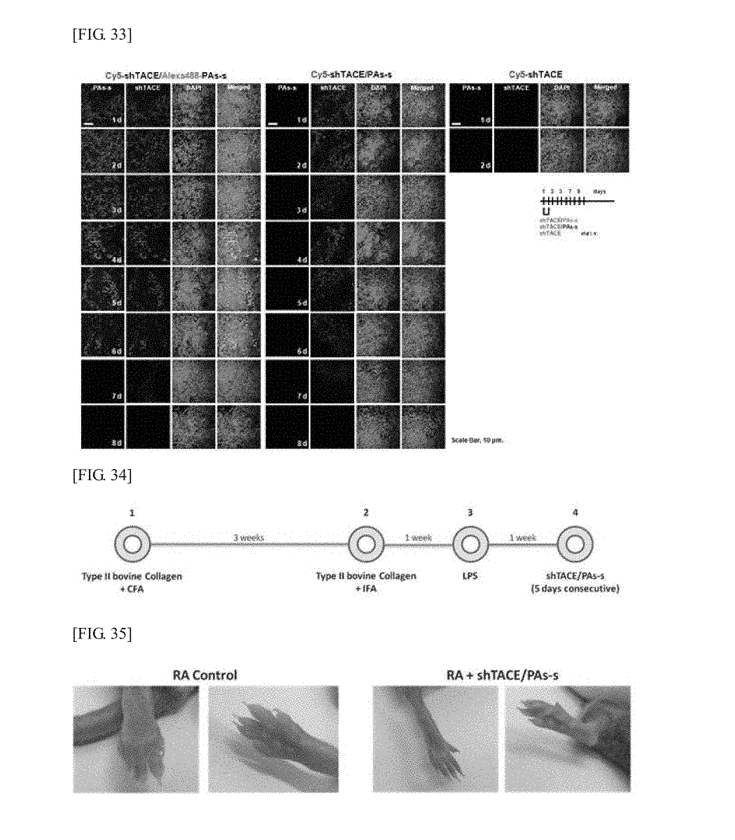

The distribution of the intravenously injected shTACE/PAs-s complex in the colon was examined [FIG. 33]. The shTACE gene was linked to Cy5, a red-fluorescent dye, and the PAs-s carrier was linked to Alexa 488 dye, a green-fluorescent dye, to obtain fluorescent-emitting gene and carrier. Then, the dye-linked gene and the dye-linked carrier were mixed and incubated for 30 minutes to form a complex. For the (Cy5-shTACE)/(Alexa488-PAs-s) complex used for intravenous infusion, 20 μg of shTACE and 40 μg of the PAs-s carrier were used according to the optimal weight ratio. The complex was intravenously injected for two consecutive days, and then the distribution of the complex was examined in the colon for 8 days. As a result, strong fluorescence was observed in the colon up to 3 days after the injection of the complex, and the maximum fluorescence was observed on the 3rd day. Thereafter, the intensity of fluorescence gradually decreased, and fluorescence was observed in the colon until the 7th day. These results confirmed that the gene/carrier complex was present in the colon for anti-inflammatory action. On the other hand, fluorescence was not observed in cells when only the shTACE gene linked to Cy5 was treated without the carrier. This is due to the enhanced permeability and retention (EPR) effect. The effect is that blood vessel walls in a cancerous state are loosened and nano-sized drugs circulating in the blood pass through the blood vessel wall and accumulate in the cancer tissues. This effect may be applied not only to cancer, but also to inflammatory ulcerative colitis. In an inflammatory state, inflammation-related substances are secreted, tissue walls are loosened by the substances, and the shTACE/PAs-s complex passes through the loosened tissue wall. Consequently, the complex is accumulated in the colon.

8. Evaluation of Therapeutic Effect of shTACE/PAs-s Complex in Rheumatoid Arthritis Model

DBA/1 female mice were used to prepare an animal model of rheumatoid arthritis. First, bovine type II collagen was mixed with CFA and injected into the tail of the mouse. After 3 weeks, bovine type II collagen was mixed with IFA and injected into the tail. One week later, LPS was injected into the peritoneal cavity to complete rheumatoid arthritis modeling [FIG. 34]. One week after injection of LPS, the gene/carrier complex (shTACE/PAs-s peptoplex) was intravenously injected for 5 consecutive days. At this time, according to the optimal weight ratio of 1:2, 20 μg of the gene and 40 μg of the carrier were mixed in PBS and incubated for 30 minutes to form a complex. Then, the complex was intravenously injected. This corresponds to treating the complex in an amount of 1 mg/kg per animal in a single injection.

After completion of rheumatoid arthritis modeling using DBA/1 mice, the complex (shTACE/PAs-s peptoplex) was injected into the mice for 5 consecutive days, and the forepaws and hind paws of the experimental animals were observed after 3 days. As a result, compared to a rheumatoid arthritis control group, which showed severe swelling of forepaws and hind paws due to inflammation, the degree of swelling in the group treated with the complex was not severe. These results confirmed that rheumatoid arthritis was alleviated by the administration of the complex [FIG. 35].

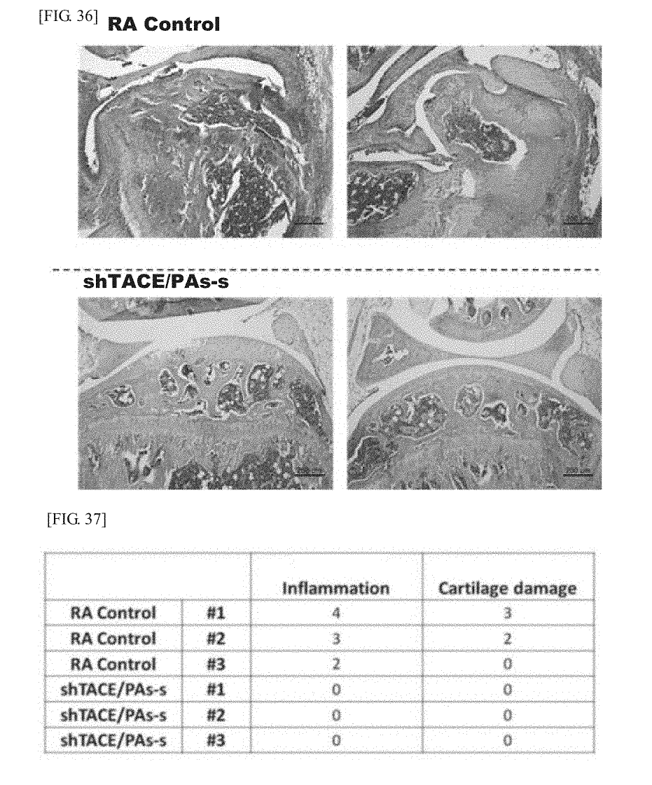

After completion of rheumatoid arthritis modeling using DBA/1 mice, the complex (shTACE/PAs-s peptoplex) was injected into the mice for 5 consecutive days, and after 3 days, the parts of the hind paw and the thigh of experimental animals were obtained, and specimens were prepared for histological analysis. The specimens were stained with H&E by a skilled pathologist, and rheumatoid arthritis related scales were measured through a blind test and scored [FIG. 36]. Inflammation increases the penetration of macrophages, neutrophils, and other immune cells into tissues and activates the immune cells. The degree of inflammation may be indirectly determined based on the degree of penetration of immune cells and the location of the penetrated tissue. The penetration of immune cells was very low in the group treated with the gene therapy agent as compared with a control group of rheumatoid arthritis in which immune cells infiltrated into the synovial membrane or the cavitas synovialis. These results indicate that inflammation is alleviated. In addition, the degree of cartilage destruction was much lower than that of the control without any treatment [FIG. 37].

### Experimental Example 2: Preparation of shTACE/PAs-s Complex and Evaluation of Efficacy Thereof

### Experimental Procedure

### <Agarose Gel Electrophoresis>

1 μg of TACE short hairpin RNA (TACE shRNA or shTACE) [SEQ ID NO: 1] was mixed with various amounts of an 8D16R carrier and incubated at room temperature for 30 minutes to form a gene/carrier complex (shTACE/8D16R peptoplex). Then, to confirm the formation of the complex, the complexes were loaded onto a 0.8% (wt/vol) agarose gel immersed in a 0.5×TBE buffer solution and subjected to electrophoresis at 100 V for 20 minutes.

### <Measurement of Surface Charge and Size of Complex>

5 μg of TACE short hairpin RNA (TACE shRNA or shTACE) [SEQ ID NO: 1] was mixed with the 8D16R carrier at a weight ratio of 4 (8D16R/shTACE=4) to form a shTACE/8D16R complex, and the total volume was adjusted to 800 μl with a buffer solution. The surface charge and size of the complex were measured using Zetasizer Nano ZS (Malvern).

### <Cell Culture>

Dulbecco's Modified Eagle Medium (DMEM) and fetal bovine serum (FBS) were purchased from WelGENE Inc. (Korea). RAW 264.7 cells (mouse-derived macrophages) were purchased from the Korean Cell Line Bank, and subcultured once every two days. Cells were cultured in complete medium supplemented with 10% FBS, penicillin (100 IU/ml), and streptomycin (100 μg/ml) at 37° C. and 5% CO2 atmospheric conditions.

### <In Vitro Cytotoxicity>

Cell viability was measured using 3-(4,5-dimethylthiazol-2-yl)-2,5-diphenyltetrazolium bromide (MTT) assay. Mouse-derived macrophages (RAW 264.7 cells) were cultured in a cell culture plate at a density of 4×10⁴ cells per 1 ml of complete medium (CM). 24 hours after cell culture, the cells were treated with a gene/carrier complex. At this time, the amount or the weight ratio of the complex was adjusted according to each experimental purpose. After 24 hours, the cells were treated with 5 mg/ml of MTT and incubated at 37° C. for 4 hours. Thereafter, the complete medium was removed from the cells, and DMSO of the same amount as the amount of the complete medium was added to the cells, followed by incubation for 15 minutes. After incubation, the cell culture plate was placed in an absorbance measurement apparatus, and absorbance was measured at a wavelength of 570 nm. Based on the obtained results, relative cell viability was calculated. The principle of MTT assay is to take advantage of the ability of mitochondria to reduce MTT, a water-soluble yellow tetrazole, to a water-insoluble purple formazan(3-(4,5-dimethylthiazol-2-yl)-2, 5-diphenyltetrazolium bromide) product using NADPH, NADH, and the like. Since MTT formazan is insoluble in water, MTT formazan is dissolved in DMSO, an organic solvent, and absorbance is measured at 570 nm. At this time, the absorbance is proportional to the metabolic activity of cells. Based on the measured absorbance, relative cell viability may be evaluated.

### <In Vitro Transfection Efficiency>

The transfection efficiency of an 8D16R carrier was measured using luciferase assay. Luciferase enzyme is expressed from a luciferase plasmid (pLuci) in cells. The enzymatic activity of luciferase oxidizes luciferin to emit light energy, and thus pLuci acts as a reporter gene that indirectly indicates the expression level of a foreign gene introduced into cells.

Each well of a cell culture plate was inoculated with 4×10⁴ mouse-derived macrophages (RAW 264.7 cells) and cultured for 24 hours. After culture, the macrophages were treated with a pLuci/8D16R complex formed by mixing a luciferase plasmid (pLuci) and an 8D16R carrier. The gene/carrier complex was prepared in a plain medium. The amount of the plasmid used was 1 μg.

After 48 hours, 200 μl of 1× reporter lysis buffer was added to each well, followed by incubation at 4° C. for 15 minutes. Then, cells in each well were collected and lysed. Then, the cell lysates were subjected to centrifugation at 1,2470 g for 3 minutes to remove cell culture supernatants, and the cell lysates were mixed with a luciferase assay reagent containing a luciferase substrate. The light emitted by chemical reaction between the cell lysates and the luciferase substrate was detected using a luminometer (Berthold Detection Systems) and expressed as a relative luminescence unit (RLU). The obtained results were expressed as RLU/mg of cell protein. Protein concentration was calculated using a DC protein assay kit including a bovine serum albumin standard. A luciferase analysis kit was purchased from Promega (USA). In other words, the transfection efficiency of the carrier was determined by the degree of chemical reaction between a luciferase substrate and cell lysates.

### <RNA Isolation and RT-PCR>

Each well of a cell culture plate was inoculated with 4×10⁴ mouse-derived macrophages (RAW 264.7 cells) and cultured for 24 hours. After culture, the macrophages were treated with a gene/carrier complex (shTACE/8D16R peptoplex) and incubated for 24 hours. Then, cells were homogenized and RNA was isolated using an RNeasy Lipid Tissue Mini Kit (Qiagen). For each group, 1 μg of the isolated RNA was reacted with reverse transcriptase to synthesize cDNA complementary to RNA of each group. Then, RT-PCR was performed on cDNA using a Cyber Premix Ex Taq RT-PCR kit, and the relative mRNA levels of TACE and TNF-α were calculated based on the mRNA level of GAPDH, an endogenous control. At this time, a forward primer and a reverse primer for TACE amplification were 5′-GTACGTCGATGCAGAGCAAA-3′ (SEQ ID NO: 2) and 5′-AAACCAGAACAGACCCAACG-3′ (SEQ ID NO: 3), respectively.

### <ELISA>

Each well of a cell culture plate was inoculated with 4×10⁴ mouse-derived macrophages (RAW 264.7 cells) and cultured for 24 hours. After culture, 100 ng/ml of lipopolysaccharide (LPS) was added to each well to activate macrophages. Activated or unactivated macrophages were treated with the shTACE/8D16R complex. After 48 hours of incubation, 1 ml of cell culture fluid was obtained from each well. The cell culture fluid was subjected to centrifugation at 4° C. and 13,000 rpm for 5 minutes to separate a supernatant. Then, for each group, the amount of TNF-α, present in the supernatant, was measured using Sandwich ELISA (eBioscience). The relative amount of TNF-α was obtained by dividing the amount of TNF-α by the amount of cellular protein. The obtained results were expressed as the amount of TNF-α per mg of cellular protein.

### <Induction of Rheumatoid Arthritis Animal Model and Injection of Therapeutic Gene Complex>

DBA/1 female mice were used to prepare an animal model of rheumatoid arthritis. First, bovine type II collagen was mixed with Complete Freund's Adjuvant (CFA) at a concentration of 1 mg/ml, and 100 μl of the solution was injected into the tail of the mouse. 3 weeks later, 100 μl of a solution in which bovine type II collagen and Incomplete Freund's Adjuvant (IFA) were mixed at a concentration of 1 mg/ml was injected into the tail of the mouse. One week after injection, 50 μg of LPS per mouse was injected intraperitoneally to worsen inflammation. One week after injection of LPS, the gene/carrier complex (shTACE/8D16R peptoplex) was intravenously injected. After rheumatoid arthritis modeling was completed, the gene/carrier complex (shTACE/8D16R peptoplex) including 20 μg of shTACE and 80 μg of the gene carrier according to an optimal weight ratio of 1:4 or monoclonal antibodies (Infliximab) targeting tumor necrosis factor-α (TNF-α) as a positive control were intravenously injected for 5 consecutive days. The amount of drug administered to experimental animals was about 1 mg/kg.

### <Images of an Animal Model of Rheumatoid Arthritis>

One week after the start of rheumatoid arthritis animal modeling, mice were treated with the gene/carrier complex (shTACE/8D16R peptoplex) for 5 consecutive days. Animal experiments were completed after 5 days. The rheumatoid arthritis model mice were anesthetized using carbon dioxide. Images of the forepaws and hind paws of anesthetized experimental animals were obtained.

### <Evaluation of Histological Score for Rheumatoid Arthritis>

Parts of the hind paw and the thigh of rheumatoid arthritis model mice were removed, and specimens were prepared for histological analysis. The tissue specimens were fixed in a buffer containing 4% formalin and stained using an H&E staining method. Observation of the stained specimens was performed by a skilled pathologist through a blind test procedure. In H&E staining, H refers to hematoxylin, and E refers to eosin. Hemalum is a complex of hematein, an oxidized form of hematoxylin, and aluminum ions, and hemalum binds to DNA and stains the nucleus. Eosin stains microfibers, extracellular fibers, and the like. The degree of progression of inflammatory state and the degree of damage of the cartilage tissue were analyzed by a pathologist's blind test, and the results were scored. Detailed scores for each item are shown in Table 4 below

TABLE 4

| | Inflammation | Cartilage damage |
|---|---|---|
| 0 | no inflammation | no destruction |
| 1 | slight thickening of the lining layer | minimal erosion |
| | or some infiltrating cells in the | limited to single spots |
| | underlying layer | |
| 2 | slight thickening of the lining layer | slight to moderate erosion |
| | plus some infiltrating cells in the | in a limited area |
| | underlying layer | |
| 3 | thickening of the lining layer, an influx | more extensive erosion |
| | of cells in the underlying layer | |
| | and the presence of cells in the synovial | |
| | space | |
| 4 | synovium highly infiltrated with many | general destruction |
| | inflammatory cells | |

### Experiment Results

1. Preparation of shTACE/8D9R Complex

The 8D9R carrier was prepared according to the same procedure described in Preparation Example 2, and the carrier contained 9 arginines.

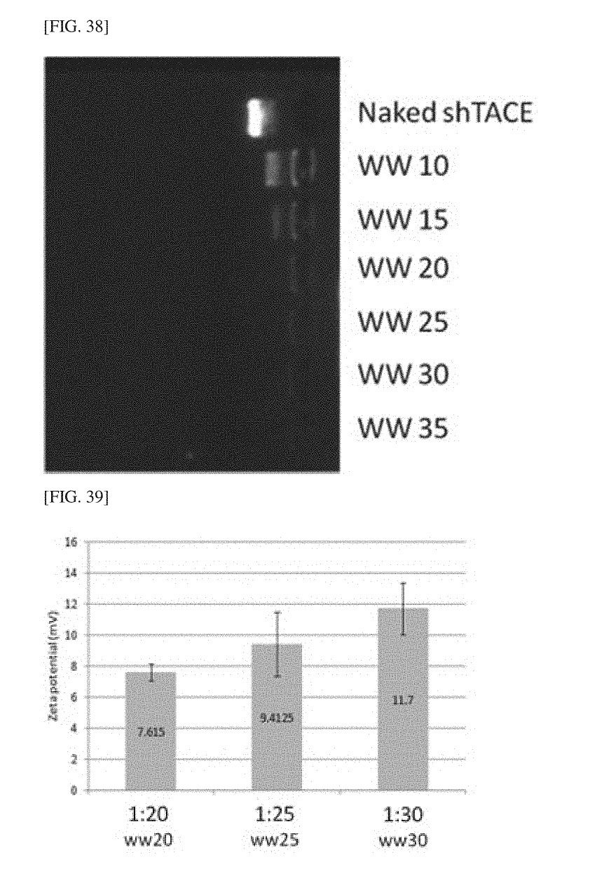

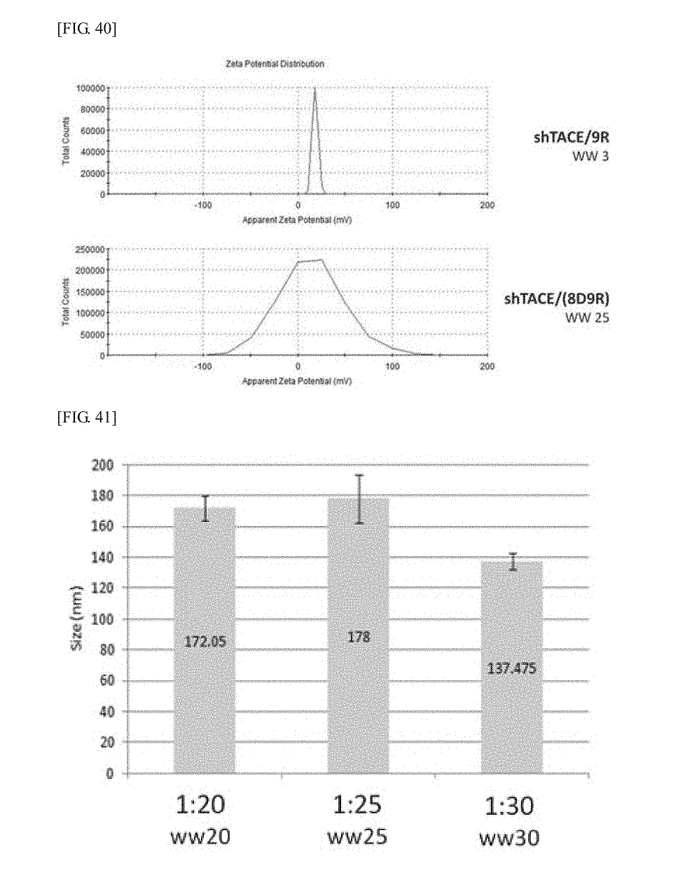

1 μg of therapeutic gene shTACE [SEQ ID NO: 1] was mixed with various amounts of the 8D9R carrier (10, 15, 20, 25, 30, and 35 μg) to form a shTACE/8D9R complex. The formation of the shTACE/8D9R complex was confirmed by electrophoresis. When the weight ratio of the carrier to the gene was 20 or more, formation of the shTACE/8D9R complex was observed [FIG. 38]. Since the overall positive charge of the carrier 8D9R was very low, a condensation effect of forming a complex by wrapping the negatively charged gene was very low. Thus, the complex was formed at a very high weight ratio. The shTACE/8D9R complex showed a low zeta potential of about 12 mV, even though the weight ratio of the carrier to the gene was as high as 30 (WW30) [FIG. 39]. In addition, referring to a zeta potential distribution diagram, shTACE/9R showed a narrow peak in a very narrow range, and the shTACE/8D9R complex showed a wide range of zeta potentials ranging from −100 mV to +100 mV [FIG. 40]. That is, in the case of the shTACE/8D9R complex, the distribution of zeta potential is observed over a wide range of negative and positive values, indicating that the condensation effect of the 8D9R carrier on the gene is low. Although the complex has an appropriate size [FIG. 41], the overall condensation effect of the carrier is lowered by the 8D peptide negatively charged in the carrier.

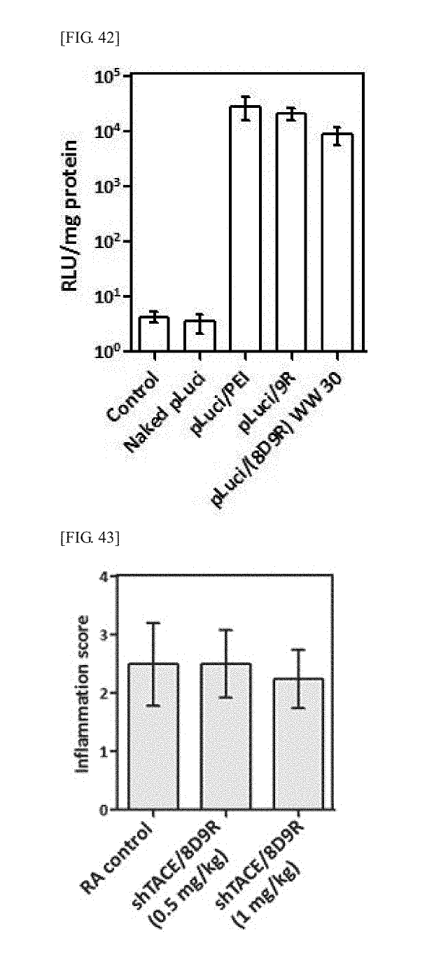

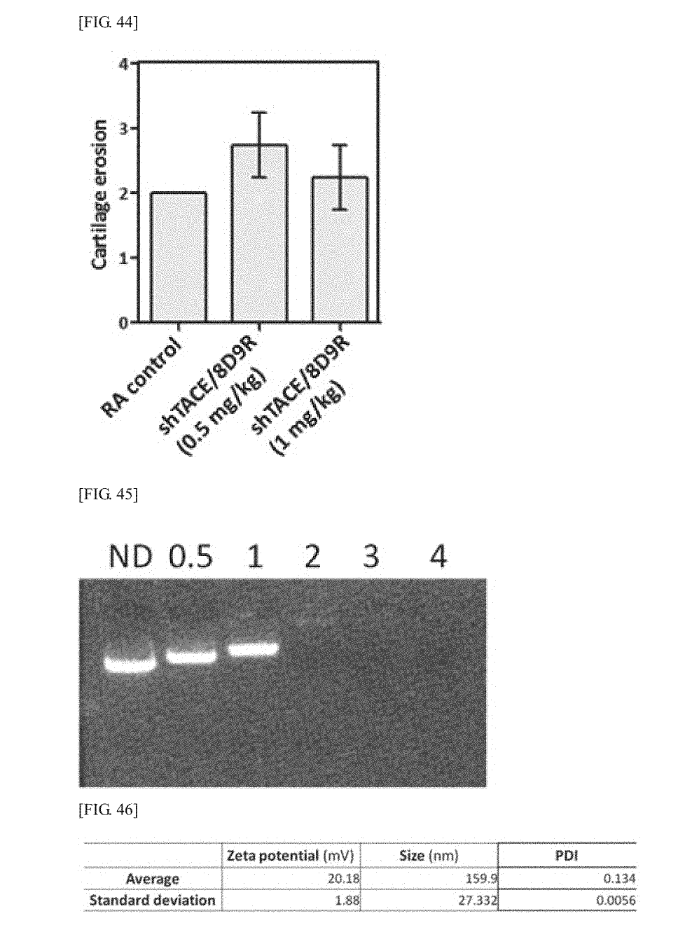

The transfection efficiency of the 8D9R carrier was also low [FIG. 42]. In addition, when the degree of inflammation progression [FIG. 43] and the degree of cartilage destruction [FIG. 44] were evaluated in a rheumatoid arthritis experimental mouse model, the shTACE/8D9R complex did not show any improvement compared to the control. These results suggest that the shTACE therapeutic gene is not efficiently delivered to cells due to the low delivery efficiency of the 8D9R carrier and as a result, the therapeutic effect of shTACE is not exhibited.

2. Optimization of shTACE/8D16R Complex and Evaluation of Anti-Inflammatory Effect Thereof

In the 8D9R carrier, the 8D peptide is a domain having a targeting ability to a bone resorption site, and the 9R peptide is a domain showing a positive charge and promotes intracellular delivery of the gene. The two domains were ligated together to synthesize a carrier capable of targeting to a bone resorption site. Arginine (Arg) is positively charged to facilitate intracellular introduction, while aspartic acid (Asp) is negatively charged, and the 8D peptide with a targeting ability also exhibits a negative charge. Thus, although there is a positively charged 9R that promotes intracellular introduction of a gene, the overall positive charge intensity of the 8D9R carrier is low due to the negatively charged 8D. As a result, when the shTACE/8D9R complex was formed, the ratio of the carrier was required to be very high to form the complex, and the ability of the carrier to wrap the gene was very low. Thus, there was little therapeutic effect when using the shTACE/8D9R complex.

Thus, the number of arginines was increased to 16 to develop an 8D16R peptide carrier.

Electrophoresis was performed to confirm whether the shTACE/8D16R complex was formed and determine the relevant weight [FIG. 45]. The total volume of the complex was adjusted. Then, the complex was loaded onto an agarose gel immersed in a 0.5×TBE buffer solution and subjected to electrophoresis at 100 V. When electrophoresis is performed, a negative charge is applied at the upper part of an agarose gel. At this time, the gene (having a negative charge) that does not form a complex with the carrier or a complex with a low ratio of gene/carrier moves to the lower part of the gel due to repulsive force. On the other hand, a complex in which the gene and the carrier are mixed in appropriate proportions does not move downward but remains in the upper part of the gel. When the weight ratio of the carrier to the gene is 2 or more, the shTACE/8D16R complex was properly formed. In addition, when the weight ratio of the carrier to the gene is 4, the complex exhibited a high zeta potential, an appropriate size, and an appropriate PDI value [FIG. 46]. Preferably, the size of the complex is about 200 nm and a PDI value is about 0.1 to 0.2.

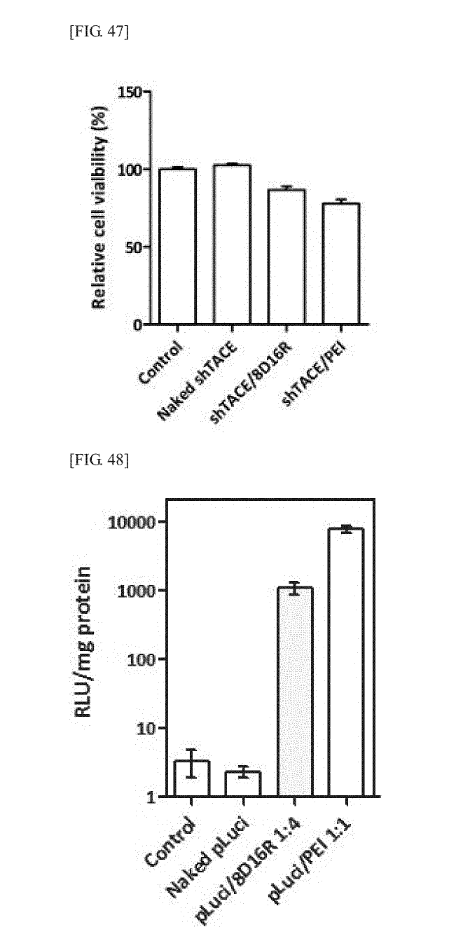

When the toxicity of the shTACE/8D16R complex was evaluated in mouse-derived macrophages (RAW 264.7 cells), the shTACE/8D16R complex having an optimal weight ratio of 4 exhibited little cytotoxicity [FIG. 47]. The shTACE/8D16R complex was formed according to the optimal weight ratio of 1:4. 24 hours after complex formation, relative cell viability was determined by MTT assay. At this time, a PEI complex was used a positive control. Compared to the PEI complex, a positive control, cells treated with the shTACE/8D16R complex exhibited high cell viability. That is, the shTACE/8D16R complex exhibited low cytotoxicity.

Transfection is a process of introducing a foreign gene into cultured animal cells and expressing the gene in cells. Transfection efficiency may be measured by transferring luciferase plasmid DNA into cells and measuring the expression level of luciferase protein. The enzymatic activity of luciferase oxidizes luciferin to emit light energy. Thus, luciferase plasmid DNA acts as a reporter gene that indirectly indicates the expression level of a foreign gene introduced into cells. 1 μg of luciferase plasmid (pLuci) was mixed with 4 μg of the 8D16R carrier and incubated at room temperature for 30 minutes to form the pLuci/8D16R complex. Then, mouse-derived macrophages (RAW 264.7 cells) were treated with the complex. When the relative reactivity with a luciferin substrate solution was measured, the 8D16R carrier showed high delivery efficiency in macrophages [FIG. 48].

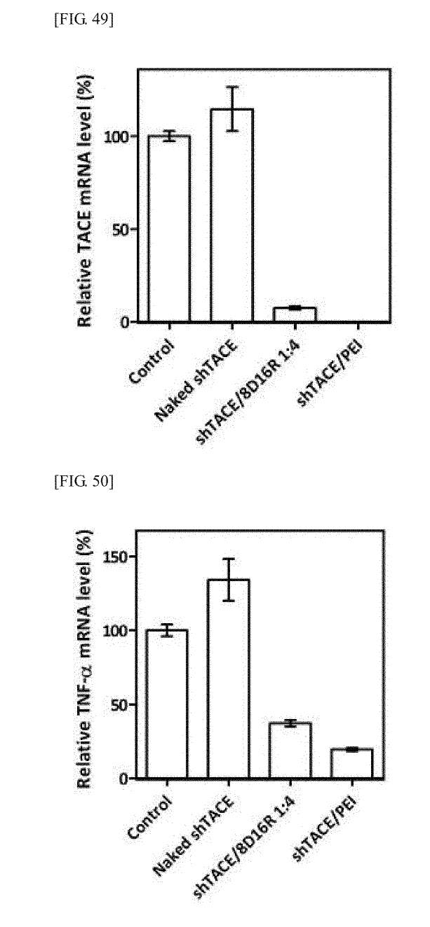

When mouse-derived macrophages were treated with the shTACE/8D16R complex, TACE expression was inhibited by the inhibitory role of shTACE [FIG. 49]. Since an RNA interference system targeting a specific gene was used in the present invention, TACE expression could be selectively regulated. RNA interference is a process by which specific RNA is degraded, resulting in suppression of gene expression. When macrophages were treated with the shTACE gene alone without the 8D16R carrier, a small amount of the negatively charged gene was introduced into the cells due to the repulsive force of the cell membrane, and thus TACE interference effect was hardly observed. On the other hand, when cells were treated with the complex including the 8D16R carrier and the shTACE gene, shTACE could efficiently enter the cell with the help of the 8D16R carrier, and thus TACE mRNA level was reduced due to the TACE interference effect of shTACE.

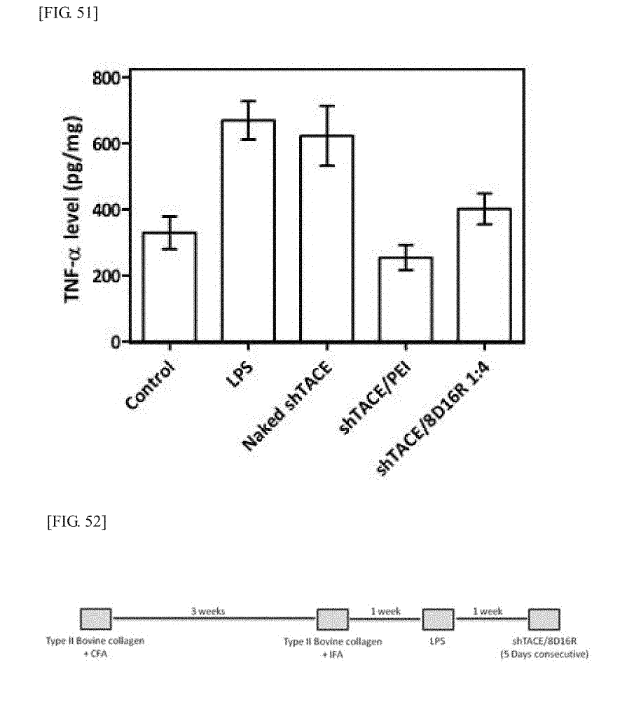

Expression of TACE is increased in a variety of inflammatory diseases and increased TACE leads to the release of TNF-α, a major inflammatory mediator, and inflammation is exacerbated by TNF-α. Thus, prevention and treatment of rheumatoid arthritis may be achieved by inhibiting TACE expression. A complex was formed by mixing short hairpin RNA (shRNA) targeting TACE and a carrier. Then, when cells were treated with the complex, the mRNA level of inflammatory cytokine TNF-α was decreased [FIG. 50]. In addition, when activated inflammatory macrophages were treated with the shTACE/8D16R complex, the amount of soluble TNF-α was also reduced [FIG. 51]. These results confirmed the anti-inflammatory effect of the shTACE/8D16R complex in macrophages.

3. Therapeutic Effect of shTACE/8D16R Complex in Rheumatoid Arthritis Mouse Model

DBA/1 female mice were used to prepare an animal model for rheumatoid arthritis. Collagen was excessively injected into the mice to induce rheumatoid arthritis, an autoimmune disease.

First, bovine-derived Type II collagen was mixed with Complete Freund's Adjuvant (CFA) and injected into mouse tail. 3 weeks later, bovine type II collagen was mixed with Incomplete Freund's Adjuvant (IFA) and injected into mouse tail. One week later, lipopolysaccharide (LPS) was injected into the peritoneal cavity to exacerbate the inflammatory reaction, completing the modeling of rheumatoid arthritis mice [FIG. 52]. One week after injection of LPS, the gene/carrier complex (shTACE/8D16R) of the present invention was intravenously injected for 5 consecutive days. According to the optimal weight ratio of 1:4, 20 μg of the gene and 80 μg of the carrier were mixed in PBS to form a complex, and the complex was intravenously injected. The amount of the complex administered to the experimental animals at a single injection was about 1 mg/kg.

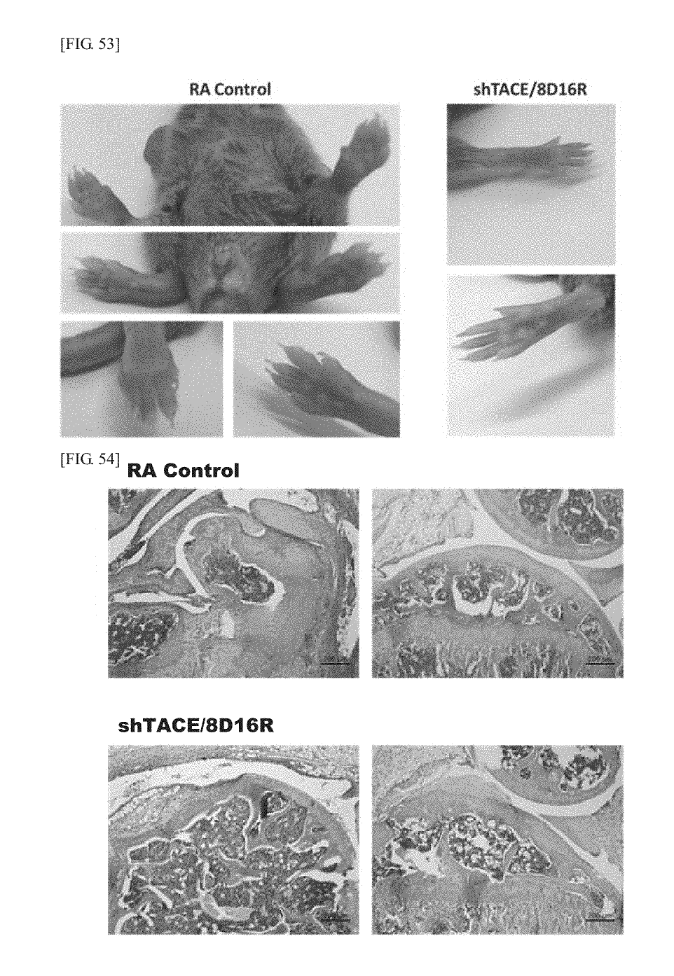

After completion of rheumatoid arthritis modeling, the complex (shTACE/8D16R) was intravenously injected for 5 consecutive days, and the forepaws and hind paws of the experimental animals were observed after 5 days. As a result, compared to a rheumatoid arthritis control group, which showed severe swelling of forepaws and hind paws due to inflammation, the degree of swelling in the group treated with the complex was not severe. These results confirmed that rheumatoid arthritis was alleviated by the administration of the complex [FIG. 53].

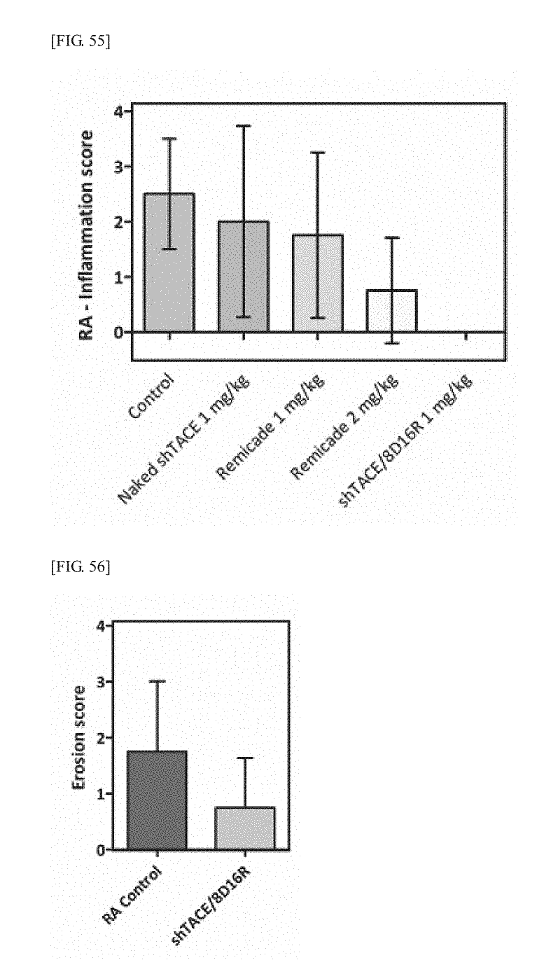

After completion of rheumatoid arthritis modeling using DBA/1 mice, the complex (shTACE/8D16R) was injected into the mice for 5 consecutive days, and after 5 days, the parts of the hind paw and the thigh of experimental animals were obtained, and specimens were prepared for histological analysis. The specimens were stained with H&E by a skilled pathologist, and rheumatoid arthritis related scales were measured through a blind test and scored [FIG. 54]. In general, inflammation increases the penetration of macrophages, neutrophils, and other immune cells into tissues and activates the immune cells. The degree of inflammation may be indirectly determined based on the degree of penetration of immune cells and the location of the penetrated tissue. The penetration of immune cells was very low in the group treated with the gene therapy agent as compared with a control group of rheumatoid arthritis in which immune cells infiltrated into the synovial membrane or the cavitas synovialis. These results indicate that inflammation is alleviated. In addition, the number of immune cells involved in inflammation decreased in the complex-treated group [FIG. 55]. In particular, the complex-treated group exhibited better efficacy than the monoclonal antibody (Infliximab, trade name: Remicade)-treated group. In this case, the monoclonal antibody is used as a positive control and targets tumor necrosis factor-α (TNF-α).

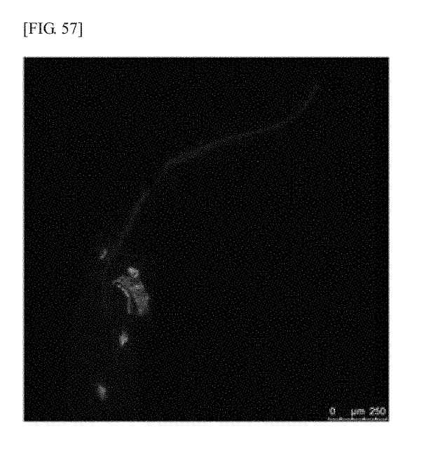

In rheumatoid arthritis, cartilage destruction occurs. The degree of cartilage destruction was very low in the shTACE/8D16R complex-treated group compared to the control group without any treatment (referring to Table 4) [FIG. 56].

8D peptides were fused to FITC fluorescence, and it was confirmed whether the carrier only targets bone resorption sites in an animal model for rheumatoid arthritis using the FITC-conjugated 8D peptides. Xylenol Orange (XO), a red color dye capable of targeting bone synthesis sites, was used as a control group.

As a result, green 8D-FITC and red XO did not overlap and were stained separately [FIG. 57]. These results demonstrate that bone synthesis sites and bone resorption sites are separately present, and that the 8D peptide used in the present invention targets only to the bone resorption sites, in an animal model of rheumatoid arthritis.

When the gene/carrier complex according to the present invention is injected into the body, therapeutic gene can be efficiently delivered, and as a result, TACE expression can be effectively inhibited. Therefore, the complex of the present invention may have an excellent effect in the prevention or treatment of inflammatory diseases.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. A gene/carrier complex comprising tumor necrosis factor-α converting enzyme (TNF-α converting enzyme, TACE) shRNA represented by SEQ ID NO: 1 and a nonviral gene carrier, wherein the nonviral gene carrier comprises an acetate of disulfide-linked poly(oligo-arginine).

2. The gene/carrier complex according to claim 1, wherein the disulfide-linked poly(oligo-arginine) is composed of nine-arginine oligomers, wherein each nine-arginine oligomer comprises disulfide-linked cysteines at both ends thereof.

3. The gene/carrier complex according to claim 1, wherein the disulfide-linked poly(oligo-arginine) comprises a repeating unit of Cys-(9×Arg)-Cys

4. The gene/carrier complex according to claim 1, wherein the disulfide-linked poly(oligo-arginine) is polymerized by disulfide crosslinking via thiol groups (—SH) of cysteines at both ends of a repeating unit.

5. The gene/carrier complex according to claim 1, wherein TACE shRNA and the gene carrier are present in a weight ratio of 1:1.5 to 8.

6. A method of preparing a gene/carrier complex, the method comprising mixing and incubating tumor necrosis factor-α converting enzyme (TNF-α converting enzyme, TACE) shRNA represented by SEQ ID NO: 1 and a nonviral gene carrier, wherein the nonviral gene carrier comprises an acetate of disulfide-linked poly(oligo-arginine).

7. The method according to claim 6, wherein the incubation is performed at 20 to 40° C. for 20 to 40 minutes.

8. The method according to claim 6, wherein the disulfide-linked poly(oligo-arginine) is composed of nine-arginine oligomers, wherein each nine-arginine oligomer comprises disulfide-linked cysteines at both ends thereof.

9. The method according to claim 6, wherein the disulfide-linked poly(oligo-arginine) comprises a repeating unit of Cys-(9×Arg)-Cys.

10. The method according to claim 6, wherein the disulfide-linked poly(oligo-arginine) is polymerized by disulfide crosslinking via thiol groups (—SH) of cysteines at both ends of a repeating unit.

11. A method of preventing or treating inflammatory diseases, the method comprising administering a therapeutic dose of the gene/carrier complex according to claim 1 to a subject.

12. The method according to claim 11, wherein the inflammatory diseases are one or more selected from the group consisting of ocular inflammation, allergic conjunctivitis, dermatitis, rhinitis, asthma, rheumatoid arthritis, acute lung injury, obesity, and inflammatory bowel disease.

13. The method according to claim 11, wherein the complex is administered through oral, aerosol, buccal, epidermal, intradermal, inhalation, intramuscular, intranasal, intraocular, intrapulmonary, intravenous, intraperitoneal, nasal, ocular, oral, ear, injection, patch, subcutaneous, hypoglossal, topical or percutaneous routes.

14. A gene/carrier complex comprising tumor necrosis factor-α converting enzyme (TNF-α converting enzyme, TACE) shRNA represented by SEQ ID NO: 1 and a nonviral gene carrier, wherein the nonviral gene carrier comprises a trifluoroacetic acid (TFA) salt of poly(oligo-aspartic acid)poly(oligo-arginine).

15. The gene/carrier complex according to claim 14, wherein the poly(oligo-aspartic acid)poly(oligo-arginine) comprises cysteines at both ends thereof.

16. The gene/carrier complex according to claim 14, wherein the poly(oligo-aspartic acid)poly(oligo-arginine) is a Cys-(8×Asp)-(16×Arg)-Cys peptide, wherein the peptide comprises cysteines at both ends thereof and is composed of an eight-aspartic acid oligomer and a sixteen-arginine oligomer.

17. The gene/carrier complex according to claim 14, wherein TACE shRNA and the gene carrier are present in a weight ratio of 1:1.5 to 8.

18. A method of preparing a gene/carrier complex, the method comprising mixing and incubating tumor necrosis factor-α converting enzyme (TNF-α converting enzyme, TACE) shRNA represented by SEQ ID NO: 1 and a nonviral gene carrier, wherein the nonviral gene carrier comprises a trifluoroacetic acid (TFA) salt of poly(oligo-aspartic acid)poly(oligo-arginine)

19. The method according to claim 18, wherein the incubation is performed at 20 to 40° C. for 20 to 40 minutes.

20. The method according to claim 18, wherein the poly(oligo-aspartic acid)poly(oligo-arginine) comprises cysteines at both ends thereof.

21. The method according to claim 18, wherein the poly(oligo-aspartic acid)poly(oligo-arginine) is a Cys-(8×Asp)-(16×Arg)-Cys peptide, wherein the peptide comprises cysteines at both ends thereof and is composed of an eight-aspartic acid oligomer and a sixteen-arginine oligomer.

22. A method of preventing or treating rheumatoid arthritis, the method comprising administering a therapeutic dose of the gene/carrier complex according to claim 14 to a subject.

23. The method according to claim 22, wherein the complex is administered through oral, aerosol, buccal, epidermal, intradermal, inhalation, intramuscular, intranasal, intraocular, intrapulmonary, intravenous, intraperitoneal, nasal, ocular, oral, ear, injection, patch, subcutaneous, hypoglossal, topical or percutaneous routes.
